(19) United States
(12) Patent Application Publication
(10) Pub. No.: US 2013/0122556 A1
(43) Pub. Date: May 16, 2013

(54) PROCESS FOR PRODUCING SULFUR-CONTAINING alpha- AMINO ACID COMPOUND

(71) Applicant: Hiroyuki Asako, Toyonaka-shi (JP)

(72) Inventor: Hiroyuki Asako, Toyonaka-shi (JP)

(73) Assignee: SUMITOMO CHEMICAL COMPANY, LIMITED, Tokyo (JP)

(21) Appl. No.: 13/806,070

(22) PCT Filed: Jul. 5, 2011
(86) PCT No.: PCT/JP2011/065748

§ 371 Date: Jan. 28, 2013

(30) Foreign Application Priority Data
Jul. 5, 2010 (JP) 2010-152779

(51) Int. Cl. C12P 13/12

(52) U.S. Cl. CPC C12P 13/12
USPC 435/113; 435/320.1; 435/252.33; 435/471

(57) ABSTRACT

The present invention provides a novel process for producing a sulfur-containing α-amino acid compounds such as methionine. A process for producing a sulfur-containing α-amino acid compound (Compound 2) comprising a step of reacting a specific sulfur-containing amino alcohol compound (Compound 1) with the following transformants and others: <Transformants> (a) both a transformant in which has been introduced into a microorganism cell a gene of an enzyme capable of converting the Compound 1 into a corresponding sulfur-containing α-amino aldehyde compound (Compound 3) (Capability A) and a transformant in which has been introduced into a microorganism cell a gene of an enzyme capable of converting the Compound 3 into a corresponding Compound 2, (b) a transformant in which has been introduced into a microorganism cell both a gene of an enzyme which has Capability A and a gene of an enzyme which has Capability B, or (c) a transformant in which has been introduced into a microorganism cell a gene of an enzyme which has both Capability A and Capability B.

### TECHNICAL FIELD

The present invention relates to a process for producing a sulfur-containing α-amino acid compound.

### BACKGROUND ART

Hitherto, methionine, which is one of sulfur-containing α-amino acid compounds, has been used as an animal feed additive. In a process for producing methionine, acrolein and methyl mercaptan are reacted with each other to produce 3-methylthiopropionaldehyde, and then the 3-methylthiopropionaldehyde obtained is reacted with hydrogen cyanide, ammonia and carbon dioxide to produce 5-(2-methyl-mercaptoethyl)-hydantoin (that is, methionine hydantoin). The resulting product is hydrolyzed under an alkaline condition to give alkali metal methionate, followed by neutralization with an acid such as sulfuric acid or carbonic acid, to liberate methionine (see, for example, JP 55-102557 A).

### DISCLOSURE OF INVENTION

The above-mentioned process employs hydrogen cyanide as C1-building block and acrolein as C3-building block, which require careful safety control in handling and an equipment adopted to such control. Accordingly, there has been demand for a novel process for producing a sulfur-containing α-amino acid compound such as methionine.

An object of the present invention is to provide a novel process for producing a sulfur-containing α-amino acid compound such as methionine.

The present invention provides:

[1] A process for producing a sulfur-containing α-amino acid compound represented by the formula (2) (hereinafter, sometimes referred to as “the Compound (2)”):

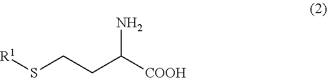

wherein R¹ represents hydrogen, an alkyl group having 1 to 8 carbon atoms, or an aryl group having 6 to 20 carbon atoms;
comprising

a step of reacting a compound represented by the formula (1) (hereinafter, sometimes referred to as “the Compound (1)”):

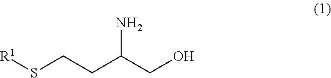

wherein R¹ is the same as defined above;
with a microbial cell or a processed product of the microbial cell of any of the following transformants (hereinafter, the transformants will be sometimes referred to as the “the present transformants”, and the process of the item [1] will be sometimes referred to as “the process of the present invention”):
(a) both
a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound (hereinafter, the enzyme will be sometimes referred to as “the present enzyme (A)”, and the polynucleotide will be sometimes referred to as “the present polynucleotide (A)”, and the transformant will be sometimes referred to as “the present transformant (A)”) and
a transformant in which has been introduced into a microorganism cell a polynucleotide comprising a nucleotide sequence encoding an amino acid sequence of an enzyme that is capable of converting the above sulfur-containing α-amino aldehyde compound into a corresponding sulfur-containing α-amino acid compound (hereinafter, the enzyme will be sometimes referred to as “the present enzyme (B)”, and the polynucleotide will be sometimes referred to as “the present polynucleotide (B)”, and the transformant will be sometimes referred to as “the present transformant (B)”);
(b) a transformant in which have been introduced into a microorganism cell both
a polynucleotide comprising a nucleotide sequence encoding an amino acid sequence of an enzyme that is capable of converting the above sulfur-containing amino alcohol compound into a corresponding sulfur-containing α-amino aldehyde compound (i.e. the polynucleotide is “the present polynucleotide (A)”, and the enzyme is “the present enzyme (A)”) and
a polynucleotide comprising a nucleotide sequence encoding an amino acid sequence of an enzyme that is capable of converting the above sulfur-containing α-amino aldehyde compound into a corresponding sulfur-containing α-amino acid compound (i.e. the polynucleotide is “the present polynucleotide (B)”, and the enzyme is “the present enzyme (B)”) (hereinafter, the transformant will be sometimes referred to as “the present transformant (AB)”); or
(c) a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (hereinafter, the enzyme will be sometimes referred to as “the present enzyme (C)”, and the polynucleotide will be sometimes referred to as “the present polynucleotide (C)”, and the transformant will be sometimes referred to as “the present transformant (C)”);
[2] The process according to the item [1] wherein the transformant is a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the enzyme is “the present enzyme (C)”, and the polynucleotide is “the present polynucleotide (C)”, and the transformant is “the present transformant (C)”), wherein said enzyme comprises any of the following amino acid sequences:
a) the amino acid sequence of SEQ ID NO: 1,
b) an amino acid sequence which is encoded by a nucleotide sequence of a DNA that hybridizes to a DNA consisting of the nucleotide sequence of SEQ ID NO: 2 or 5 under a stringent condition and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, or
c) an amino acid sequence in which one or a plurality of amino acids have been deleted, substituted or added in the amino acid sequence of SEQ ID NO: 1 and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound;
[3] The process according to the item [1] or [2] wherein R¹ of the sulfur-containing amino alcohol compound and the sulfur-containing α-amino acid compound is an alkyl group having 1 to 5 carbon atoms;
[4] The process according to the item [1] or [2] wherein R¹ of the sulfur-containing amino alcohol compound and the sulfur-containing α-amino acid compound is a methyl group;
[5] A recombinant vector comprising a DNA in which a promoter functional in a microorganism cell is operably linked to a polynucleotide comprising a nucleotide sequence encoding an amino acid sequence of an enzyme that is capable of converting the above sulfur-containing amino alcohol compound represented by the formula (1):

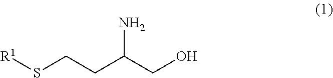

wherein R¹ represents hydrogen, an alkyl group having 1 to 8 carbon atoms, or an aryl group having 6 to 20 carbon atoms;
into a corresponding sulfur-containing α-amino aldehyde compound as well as capable of converting the above sulfur-containing α-amino aldehyde compound into a corresponding sulfur-containing α-amino acid compound (i.e. the enzyme is “the present enzyme (C)”, and the polynucleotide is “the present polynucleotide (C)”);
[6] The vector according to the item [5] wherein the polynucleotide comprises the nucleotide sequence of SEQ ID NO: 5;
[7] A transformant in which the recombinant vector of the item [5] or [6] has been introduced into a microorganism cell;
[8] The transformant according to the item [7] wherein the microorganism cell is an E. coli cell;
[9] A process for producing a transformant comprising a step of introducing the vector of the item [5] or [6] into a microorganism cell;
[10] A use of a microbial cell or a processed product of the microbial cell of any of the following transformants as a catalyst for converting a sulfur-containing amino alcohol compound represented by the formula (1):

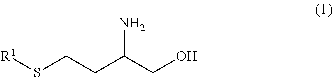

wherein R¹ represents hydrogen, an alkyl group having 1 to 8 carbon atoms, or an aryl group having 6 to 20 carbon atoms;
into a corresponding sulfur-containing α-amino acid compound:
(a) both
a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound (i.e. the enzyme is “the present enzyme (A)”, and the polynucleotide is “the present polynucleotide (A)”, and the transformant is “the present transformant (A)”) and
a transformant in which has been introduced into a microorganism cell a polynucleotide comprising a nucleotide sequence encoding an amino acid sequence of an enzyme capable of converting the above sulfur-containing α-amino aldehyde compound into a corresponding sulfur-containing α-amino acid compound (i.e. the enzyme is “the present enzyme (B)”, and the polynucleotide is “the present polynucleotide (B)”, and the transformant is “the present transformant (B)”),
(b) a transformant in which have been introduced into a microorganism cell both
a polynucleotide comprising a nucleotide sequence encoding an amino acid sequence of an enzyme capable of converting the above sulfur-containing amino alcohol compound into a corresponding sulfur-containing α-amino aldehyde compound (i.e. the polynucleotide is “the present polynucleotide (A)”, and the enzyme is “the present enzyme (A)”) and
a polynucleotide comprising a nucleotide sequence encoding an amino acid sequence of an enzyme capable of converting the above sulfur-containing α-amino aldehyde compound into a corresponding sulfur-containing α-amino acid compound (i.e. the polynucleotide is “the present polynucleotide (B)”, and the enzyme is “the present enzyme (B)”, and the transformant is “the present transformant (AB)”), or
(c) a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the polynucleotide is “the present polynucleotide (C)”, and the enzyme is “the present enzyme (C)”, and the transformant is “the present transformant (C)”);
[11] The use according to the item [10] wherein the transformant is a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the enzyme is “the present enzyme (C)”, and the polynucleotide is “the present polynucleotide (C)”, and the transformant is “the present transformant (C)”), wherein said enzyme comprises any of the following amino acid sequences:
a) the amino acid sequence of SEQ ID NO: 1,
b) an amino acid sequence which is encoded by a nucleotide sequence of a DNA that hybridizes to a DNA consisting of the nucleotide sequence of SEQ ID NO: 2 or 5 under a stringent condition and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, or
c) an amino acid sequence in which one or a plurality of amino acids have been deleted, substituted or added in the amino acid sequence of SEQ ID NO: 1 and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound;
[12] The use according to the item [10] or [11] wherein the transformant is a transformant in which has been introduced into a microorganism cell a polynucleotide comprising the nucleotide sequence encoding an amino acid sequence of SEQ ID NO: 1;
[13] The use according to the item [10] or [11] wherein R¹ of the sulfur-containing amino alcohol compound is an alkyl group having 1 to 5 carbon atoms;
[14] The use according to the item [10] or [11] wherein R¹ of the sulfur-containing amino alcohol compound is a methyl group;
[15] A process for producing a sulfur-containing α-amino acid compound represented by the formula (2):

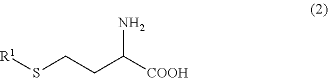

wherein R¹ represents hydrogen, an alkyl group having 1 to 8 carbon atoms, or an aryl group having 6 to 20 carbon atoms
(i.e. the compound is “the Compound (2)”);
comprising

a step of reacting a sulfur-containing amino alcohol compound represented by the formula (1):

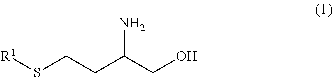

wherein R¹ is the same as defined above
(i.e. the compound is “the Compound (1)”);
with an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the enzyme is “the present enzyme (C)”);
[16] A process for producing a sulfur-containing α-amino acid compound represented by the formula (2):

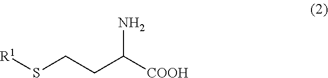

wherein R¹ represents hydrogen, an alkyl group having 1 to 8 carbon atoms, or an aryl group having 6 to 20 carbon atoms
(i.e. the compound is “the Compound (2)”);
comprising

a step of reacting a sulfur-containing amino alcohol compound represented by the formula (1):

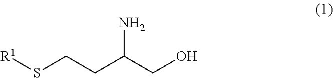

wherein R¹ is the same as defined above
(i.e. the compound is the above “the Compound (1)”);
with an enzyme capable of converting the above sulfur-containing amino alcohol compound into a corresponding sulfur-containing α-amino aldehyde compound (i.e. the enzyme is “the present enzyme (A)”), and

a step of reacting the sulfur-containing α-amino aldehyde compound obtained in the above step with an enzyme capable of converting the above sulfur-containing α-amino aldehyde compound into a corresponding sulfur-containing α-amino acid compound (i.e. the enzyme is “the present enzyme (B)”);

[17] The process according to the item [15] or [16] wherein R¹ of the sulfur-containing amino alcohol compound and the sulfur-containing α-amino acid compound is an alkyl group having 1 to 5 carbon atoms; and
[18] The process according to the item [15] or [16] wherein R¹ of the sulfur-containing amino alcohol compound and the sulfur-containing α-amino acid compound is a methyl group.

The present invention is capable of providing a novel process for producing a sulfur-containing α-amino acid compound such as methionine.

### MODE FOR CARRYING OUT THE INVENTION

It will be understood that the inventions described is not limited to the particular methodologies, protocols, and reagents described herein and that they can be modified. It will be understood that the terms used herein are meant only to describe a particular embodiment of the present invention, and that such terms do not limit the scope of the present invention.

Unless otherwise noted, all of the technical terms and chemical terms used herein have the same meanings as those commonly understood by a person skilled in the technical field of the present invention. While the present invention may be carried out or examined by using methods or materials similar or equivalent to those described herein, some of the preferred methods, equipments, and materials are described in the following.

Hereinafter, the present invention is explained in more detail.

The process of the present invention comprises:

a step of reacting a sulfur-containing amino alcohol compound represented by the formula (1):

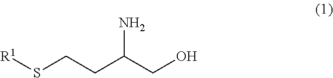

wherein R¹ represents hydrogen, an alkyl group having 1 to 8 carbon atoms, or an aryl group having 6 to 20 carbon atoms
(i.e. the compound is “the present Compound (1)”);
with a microbial cell or a processed product of the microbial cell of any of the following transformants (i.e. the transformant is “the present transformant”):

### <Transformants>

(a) both a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound (i.e. the enzyme is “the present enzyme (A)”, and the polynucleotide is “the present polynucleotide (A)”, and the transformant is “the present transformant (A)”) and a transformant in which has been introduced into a microorganism cell a polynucleotide comprising a nucleotide sequence encoding an amino acid sequence of an enzyme that is capable of converting the above sulfur-containing α-amino aldehyde compound into a corresponding sulfur-containing α-amino acid compound (i.e. the enzyme is “the present enzyme (B)”, and the polynucleotide is “the present polynucleotide (B)”, and the transformant is “the present transformant (B)”).

Examples of “an alkyl group having 1 to 8 carbon atoms” represented by R¹ in Compound (1) and Compound (2) include a methyl group, an ethyl group, a propyl group, an isopropyl group, a butyl group, an isobutyl group, a t-butyl group, a pentyl group, a hexyl group, a heptyl group, and an octyl group. Examples of “an aryl group having 6 to 20 carbon atoms” represented by R¹ include a phenyl group, a tolyl group, and a naphthyl group.

Preferred examples of R¹ include an alkyl group having 1 to 8 carbon atoms. More preferred examples of R¹ include an alkyl group having 1 to 5 carbon atoms. Further more preferred examples of R¹ include a methyl group.

A method for obtaining a microbial cell or a processed product of the microbial cell of the present transformant as a catalyst to be used in the process of the present invention is described.

In a first method,

(a) a polynucleotide comprising a nucleotide sequence encoding an amino acid sequence of an enzyme that is capable of converting the sulfur-containing amino alcohol compound to the corresponding sulfur-containing α-amino aldehyde compound (i.e. the enzyme is “the present enzyme (A)”, and the polynucleotide is “the present polynucleotide (A)”) is introduced into a microorganism cell by using a conventional genetic engineering procedure to produce the present transformant (A), and also separately, a polynucleotide comprising a nucleotide sequence encoding an amino acid sequence of an enzyme that is capable of converting the above sulfur-containing α-aldehyde compound to the corresponding sulfur-containing α-amino acid compound (i.e. the enzyme is “the present enzyme (B)” and the polynucleotide is “the present polynucleotide (B)”.) is introduced into a microorganism cell by using a conventional genetic engineering procedure to produce the present transformant (B), which thus results in a production of both transformants (i.e. “the present transformant (A)” and “the present transformant (B)”).

In a second method,

(b) a polynucleotide comprising a nucleotide sequence encoding an amino acid sequence of an enzyme that is capable of converting the above sulfur-containing amino alcohol compound into a corresponding sulfur-containing α-amino aldehyde compound (i.e. the enzyme is “the present enzyme (A)”, and the polynucleotide is “the present polynucleotide (A)”) and a polynucleotide comprising a nucleotide sequence encoding an amino acid sequence of an enzyme that is capable of converting the above sulfur-containing α-amino aldehyde compound into a corresponding sulfur-containing α-amino acid compound (i.e. the enzyme is “the present enzyme (B)”, and the polynucleotide is “the present polynucleotide (B)”) are both introduced into a microorganism cell by using a conventional genetic engineering procedure to produce a transformant (i.e. “the present transformant (AB)”).

In a third method,

(c) a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the enzyme is “the present enzyme (C)”, and the polynucleotide is “the present polynucleotide (C)”) is introduced into a microorganism cell by using a conventional genetic engineering procedure to produce a transformant (i.e. “the present transformant (C)”).

Hereinafter, the enzyme (A), the enzyme (B) and the enzyme (C) are sometimes collectively-referred to as “the present enzyme”. The polynucleotide (A), the polynucleotide (B) and the polynucleotide (C) are sometimes collectively-referred to as “the present polynucleotide”. The transformant (A), the transformant (B) and the transformant (C) are sometimes collectively-referred to as “the present transformant”.

A method for producing the present transformant according to a foreign gene introduction technique is described.

Examples of the present enzyme include an enzyme capable of converting the above sulfur-containing amino alcohol compound into a corresponding sulfur-containing α-amino aldehyde compound (i.e. “the present enzyme (A)”), an enzyme capable of converting the above sulfur-containing α-amino aldehyde compound into a corresponding sulfur-containing α-amino acid compound (i.e. “the present enzyme (B)”), and an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. “the present enzyme (C)”).

Examples of the present enzyme (A) include an enzyme comprising any of the following amino acid sequences:

a) an amino acid sequence of an alcohol dehydrogenase (i.e. an enzyme that catalyzes a reaction of oxidizing an alcohol to an aldehyde),
b) an amino acid sequence which is encoded by a nucleotide sequence of a DNA that hybridizes to a DNA consisting of a nucleotide sequence encoding an amino acid sequence of an alcohol dehydrogenase under a stringent condition, and which is an amino acid sequence of an enzyme capable of converting the above sulfur-containing amino alcohol compound into a corresponding sulfur-containing α-amino aldehyde compound, or
c) an amino acid sequence in which one or a plurality of amino acids have been deleted, substituted or added in an amino acid sequence of an alcohol dehydrogenase, and which is an amino acid sequence of an enzyme capable of converting the above sulfur-containing amino alcohol compound into a corresponding sulfur-containing α-amino aldehyde compound.

Examples of the present enzyme (B) include an enzyme comprising any of the following amino acid sequences:

a) an amino acid sequence of an aldehyde dehydrogenase (that is, an enzyme that catalyzes a reaction of oxidizing an aldehyde to a carboxylic acid),
b) an amino acid sequence which is encoded by a nucleotide sequence of a DNA that hybridizes to a DNA consisting of a nucleotide sequence encoding an amino acid sequence of an aldehyde dehydrogenase under a stringent condition, and which is an amino acid sequence of an enzyme capable of converting the above sulfur-containing α-amino aldehyde compound into a corresponding sulfur-containing α-amino acid compound, or
c) an amino acid sequence in which one or a plurality of amino acids have been deleted, substituted or added in an amino acid sequence of an aldehyde dehydrogenase, and which is an amino acid sequence of an enzyme capable of converting the above sulfur-containing α-amino aldehyde compound into a corresponding sulfur-containing α-amino acid compound.

Examples of the present enzyme (C) include an enzyme comprising any of the following amino acid sequences:

a) an amino aid sequence of an aldehyde/alcohol dehydrogenase (that is, an enzyme that catalyzes both a reaction of oxidizing an alcohol to an aldehyde and a reaction of oxidizing an aldehyde to a carboxylic acid),
b) an amino acid sequence which is encoded by a nucleotide sequence of a DNA that hybridizes to a DNA consisting of a nucleotide sequence encoding an amino acid sequence of an aldehyde/alcohol dehydrogenase under a stringent condition, and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound,
c) an amino acid sequence in which one or a plurality of amino acids have been deleted, substituted or added in an amino acid sequence of an aldehyde/alcohol dehydrogenase, and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound,
d) the amino acid sequence of SEQ ID NO: 1,
e) an amino acid sequence which is encoded by a nucleotide sequence of a DNA that hybridizes to a DNA consisting of the nucleotide sequence of SEQ ID NO: 2 or 5 under a stringent condition and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, or
f) an amino acid sequence in which one or a plurality of amino acids have been deleted, substituted or added in the amino acid sequence of SEQ ID NO: 1 and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

Examples of the present polynucleotide include:

(a) a polynucleotide comprising a nucleotide sequence encoding an amino acid sequence of an enzyme capable of converting the above sulfur-containing amino alcohol compound into a corresponding sulfur-containing α-amino aldehyde compound (i.e. the enzyme is the present enzyme (A), and the polynucleotide is the present polynucleotide (A)),
(b) a polynucleotide comprising a nucleotide sequence encoding an amino acid sequence of an enzyme capable of converting the above sulfur-containing α-amino aldehyde compound into a corresponding sulfur-containing α-amino acid compound (i.e. the enzyme is the present enzyme (B), and the polynucleotide is the present polynucleotide (B)), and
(c) a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the sulfur-containing α-amino aldehyde compound into a corresponding sulfur-containing α-amino acid compound (i.e. the enzyme is the present enzyme (C), and the polynucleotide is the present polynucleotide (C)).

Specific examples of the present polynucleotide (C) include a DNA comprising the nucleotide sequence of SEQ ID NO: 2 (a nucleotide sequence of a gene of an enzyme from bovine liver) and a DNA comprising the nucleotide sequence of SEQ ID NO: 5 (a nucleotide sequence designed by selecting a codon that is frequently used in E. coli among codons coding an amino acid that is included in an amino acid sequence of an enzyme from horse liver).

Examples of the present polynucleotide may include a natural gene, or a gene generated by introducing a mutation into a natural gene with site-directed mutagenesis or mutagenesis-treatment and others. When screening a natural gene, it may be advantageous as a subject to be microorganisms capable of producing an enzyme capable of converting the above sulfur-containing amino alcohol into a corresponding sulfur-containing α-amino aldehyde compound (i.e. the present enzyme (A)), a microorganism capable of producing an enzyme capable of converting the above sulfur-containing α-amino aldehyde into a corresponding sulfur-containing α-amino acid compound (i.e. the present enzyme (B)), and a microorganism capable of producing an enzyme that is capable of converting the above sulfur-containing amino alcohol into a corresponding sulfur-containing α-amino aldehyde compound as well as capable of converting the above sulfur-containing α-amino aldehyde into a corresponding sulfur-containing α-amino acid compound (i.e. the present enzyme (C)).

Examples of the microorganism as a population for screening a microorganism capable of producing the above enzyme include one or more microorganisms selected from a group consisting of microorganisms of the genus Alcaligenes, microorganisms of the genus Bacillus, microorganisms of the genus Pseudomonas, microorganisms of the genus Rhodobacter and microorganisms of the genus Rhodococcus.

Examples of the microorganism as a population for screening a microorganism capable of producing the above enzyme also include one or more microorganisms selected from a group consinting of Alcaligenes denitrificans, Alcaligenes eutrophus, Alcaligenes faecalis, Alcaligenes sp., Alcaligenes xylosoxydans, Bacillus alvei, Bacillus badius, Bacillus brevis, Bacillus cereus, Bacillus coagulans, Bacillus firmus, Bacillus licheniformis, Bacillus moritai, Bacillus pumilus, Bacillus sphaericus, Bacillus subtilis, Bacillus validus, Pseudomonas denitrificans, Pseudomonas ficuserectae, Pseudomonas fragi, Pseudomonas mendocina, Pseudomonas oleovorans, Pseudomonas ovalis, Pseudomonas pseudoalcaligenes, Pseudomonas putida, Pseudomonas putrefaciens, Pseudomonas riboflavina, Pseudomonas straminea, Pseudomonas syringae, Pseudomonas tabaci, Pseudomonas taetrolens, Pseudomonas vesicularis, Rhodobacter sphaeroides, Rhodococcus erythropolis, Rhodococcus groberulus, Rhodococcus rhodochrous, and Rhodococcus sp.

Preferred examples of the microorganism as a population for screening a microorganism capable of producing the above enzyme include one or more microorganisms selected from a group consisting of Alcaligenes denitrificans JCM5490, Alcaligenes eutrophus ATCC43123, Alcaligenes faecalis IFO12669, Alcaligenes sp. IFO14130, Alcaligenes xylosoxydans IFO15125t, Alcaligenes xylosoxydans IFO15126t, Bacillus alvei IFO3343t, Bacillus badius ATCC14574t, Bacillus brevis JCM2503t, Bacillus cereus JCM2152t, Bacillus coagulans JCM2257t, Bacillus firmus JCM2512t, Bacillus licheniformis ATCC27811, Bacillus licheniformis IFO12197, Bacillus licheniformis IFO12200t, Bacillus moritai ATCC21282, Bacillus pumilus IFO12092t, Bacillus sphaericus IFO3341, Bacillus sphaericus IFO3526, Bacillus subtilis ATCC14593, Bacillus subtilis ATCC15841, Bacillus subtilis IFO3108, Bacillus subtilis IFO3132, Bacillus subtilis IFO3026, Bacillus subtilis IFO3037, Bacillus subtilis IFO3108, Bacillus subtilis IFO3134, Bacillus validus IFO13635, Pseudomonas denitrificans IAM1426, Pseudomonas denitrificans IAM1923, Pseudomonas ficuserectae JCM2400t, Pseudomonas fragi IAM12402, Pseudomonas fragi IFO3458t, Pseudomonas mendocina IFO14162, Pseudomonas oleovorans IFO13583t, Pseudomonas ovalis IFO12688, Pseudomonas pseudoalcaligenes JCM5968t, Pseudomonas putida IFO12996, Pseudomonas putida IFO14164t, Pseudomonas putida IFO3738, Pseudomonas putida IFO12653, Pseudomonas putrefaciens IFO3910, Pseudomonas riboflavina IFO13584t, Pseudomonas straminea JCM2783t, Pseudomonas syringae IFO14055, Pseudomonas tabaci IFO3508, Pseudomonas taetrolens IFO3460, Pseudomonas vesicularis JCM1477t, Rhodobacter sphaeroides ATCC17023, Rhodococcus erythropolis IFO12320, Rhodococcus groberulus ATCC15076, Rhodococcus rhodochrous ATCC15076, Rhodococcus rhodochrous ATCC15610, Rhodococcus rhodochrous ATCC19067, Rhodococcus rhodochrous ATCC19149, Rhodococcus rhodochrous ATCC19150, Rhodococcus rhodochrous ATCC21197, Rhodococcus rhodochrous ATCC21199, Rhodococcus rhodochrous JCM3202t, Rhodococcus sp. ATCC19070, Rhodococcus sp. ATCC19071 and Rhodococcus sp. ATCC19148.

These microorganisms may be either isolated from natural sources, or easily gotten by purchasing from culture collection.

Examples of culture collection from which the microorganisms can be purchased, include the following culture collections.

### 1. IFO (Institute of Fermentation Osaka) Culture Collection

At present, the culture collection is transferred to National Institute of Technology and Evaluation Biological Resource Center (NBRC). Microorganisms can be purchased by filing an application to NBRC, which can be done by, for example, accessing the website of NBRC (http://www.nbrc.nite.go.jp/NBRC2/NBRCDispSearchServlet?lang=jp).

### 2. ATCC (American Type Culture Collection)

Microorganisms can be purchased through Summit Pharmaceuticals International Corporation, ATCC Industry Division by, for example, accessing its website (http://www.summitpharma.co.jp/japanese/service/s_ATCC.html). Alternatively, microorganisms can be purchased directly from ATCC.

### 3. JCM (Japan Collection of Microorganisms)

At present, the culture collection is transferred to National Institute of Physical and Chemical Research Biological Resource Center (RIKEN BRC), Microbe Division. Microorganisms can be purchased by filing an application to RIKEN BRC, which can be done by, for example, accessing a site for the culture collection in the website of RIKEN (http://www.jcm.riken.go.jp/JCM/aboutJCM_J.shtml).

### 4. IAM Culture Collection

At present, among the IAM Culture Collection, bacteria, yeasts, and filamentous fungi are transferred to National Institute of Physical and Chemical Research Biological Resource Center, Microbe Division (JCM), and microalgae are transferred to Microbial Culture Collection in National Institute for Environmental Studies (NIES). Microorganisms can be purchased by filing an application to JCM or NIES, which can be done by, for example, accessing a site for the culture collection in the website of JCM (http://www.jcm.riken.go.jp/JCM/aboutJCM_J.shtml) or in the website of NIES (http://mcc.nies.go.jp/aboutOnlineOrder.do).

As specific examples of screening the above microorganism capable of producing the above enzyme, in a test tube is placed 5 ml of sterilized culture medium, and thereto is inoculated with a microorganism obtained by purchasing from a culture collection or a microorganism isolated from soil. The resultant is incubated with shaking at 30° C. under an aerobic condition. After the completion of the incubation, the microbial cells are collected by centrifugation to obtain a viable cells. In a screw-top test tube is placed 2 ml of 0.1 M Tris-glycine buffer (pH 10), and thereto are added the above-prepared viable cells, and the mixture is suspended. To the suspension is added 2 mg of a sulfur-containing amino alcohol compound such as methioninol and/or a sulfur-containing α-amino aldehyde compound such as 1-amino-3-(methylthio)propylaldehyde, and the resulting mixture is shaken at 30° C. for 3 to 7 days.

After the completion of the reaction, 1 ml of the reaction solution is sampled. The cells are removed from the solution sample, and thereafter, the amount of the produced a sulfur-containing α-amino aldehyde compound such as 1-amino-3-(methylthio)propylaldehyde and/or a sulfur-containing α-amino acid compound such as methionine is analyzed by liquid chromatography.

Thus, microorganisms capable of preferentially oxidizing the hydroxyl group of the above sulfur-containing amino alcohol compound and/or capable of preferentially oxidizing the aldehyde group of the sulfur-containing α-amino aldehyde compound are screened.

Then, from the screened microorganism, a polynucleotide comprising a nucleotide sequence encoding an amino acid sequence of microorganism that catalyzes a desired reaction, that is, the polynucleotide comprising the nucleotide sequence encoding the amino acid sequence of the enzyme capable of converting the above sulfur-containing amino alcohol compound into a corresponding sulfur-containing α-amino aldehyde compound (i.e. the enzyme is the present enzyme (A), and the polynucleotide is the present polynucleotide (A)), the polynucleotide comprising the nucleotide sequence encoding the amino acid sequence of the enzyme capable of converting the above sulfur-containing α-amino aldehyde compound into a corresponding sulfur-containing α-amino acid compound (i.e. the enzyme is the present enzyme (B), and the polynucleotide is the present polynucleotide (B)), or the polynucleotide comprising the nucleotide sequence encoding the amino acid sequence of the enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound (i.e. the enzyme is the present enzyme (C), and the polynucleotide is the present polynucleotide (C)).

Among the present polynucleotide, the polynucleotide (A) comprises a nucleotide sequence encoding an amino acid sequence of an enzyme capable of converting the above sulfur-containing amino alcohol compound into a corresponding sulfur-containing α-amino aldehyde compound. The polynucleotide (B) comprises a nucleotide sequence encoding an amino acid sequence of an enzyme capable of converting the above sulfur-containing α-amino aldehyde compound into a corresponding sulfur-containing α-amino acid compound. The polynucleotide (C) comprises a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

“The DNA that hybridizes to a DNA consisting of a nucleotide sequence encoding an amino acid sequence of alcohol dehydrogenase under a stringent condition” in the present polynucleotide (A), “the DNA that hybridizes to a DNA consisting of a nucleotide sequence encoding an amino acid sequence of aldehyde dehydrogenase under a stringent condition” in the present polynucleotide (B), or “the DNA that hybridizes with a DNA consisting of a nucleotide sequence encoding an amino acid sequence of aldehyde/alcohol dehydrogenase under a stringent condition” in the present polynucleotide (C) denotes DNA that (1) forms a DNA-DNA hybrid with (A) a DNA having a nucleotide sequence encoding an amino acid sequence of alcohol dehydrogenase, (B) a DNA having a nucleotide sequence encoding an amino acid sequence of aldehyde dehydrogenase, or (C) a DNA having a nucleotide sequence encoding an amino acid sequence of aldehyde/alcohol dehydrogenase by hybridizing at 65° C. under high ion concentration condition [for example, 6×SSC (900 mM sodium chloride, 90 mM sodium citrate)], and (2) the resulting hybrid can be maintained even after temperature insulation at 65° C. for 30 minutes under low ion concentration condition [for example, 0.1×SSC (15 mM sodium chloride, 1.5 mM sodium citrate)], in the Southern Hybridization techniques, for example, described in “Cloning and Sequence” (Itaru Watanabe supervised, Masahiro Sugiura edited, 1989, published by Noson Bunka shay, “Molecular Cloning, A Laboratory Manual 2nd ed.” (Cold Spring Harbor Laboratory Press (1989)), “Current Protocols in Molecular Biology” (John Wiley & Sons (1987-1997)) and others.

Specific examples include (a) DNA comprising a nucleotide sequence encoding an amino acid sequence of alcohol dehydrogenase; (b) DNA comprising a nucleotide sequence encoding an amino acid sequence of aldehyde dehydrogenase; (c) DNA which comprises a nucleotide sequence encoding an amino acid sequence of aldehyde/alcohol dehydrogenase, the nucleotide sequence having a sequence homology of 80% or more, 90% or more, 95% or more, 98% or more, or 99% or more with the nucleotide sequence of SEQ ID No: 2; (d) DNA which comprises a nucleotide sequence encoding an amino acid sequence of aldehyde/alcohol dehydrogenase, the amino acid sequence having a sequence homology of 80% or more, 90% or more, 95% or more, 98% or more, or 99% or more with the amino acid sequence of SEQ ID No: 1; or DNA comprising a nucleotide sequence containing deletion, substitution or addition of partial nucleotide bases, and others. Such DNA may be DNA cloned from DNAs present in the natural field, DNA containing artificially introduced deletion, substitution or addition of partial nucleotide bases in a nucleotide sequence of this cloned DNA, or artificially synthesized DNA.

More specific examples of a nucleotide sequence in which one or a plurality of amino acids have been deleted, substituted or added in the nucleotide sequence of SEQ ID NO: 2 or 5 include: (i) a nucleotide sequence wherein 1 to 10 (for example, 1 to 5, preferably 1 to 3, more preferably 1 to 2) nucleotide base(s) is/are deleted in the nucleotide sequence of SEQ ID No: 2 or 5, (ii) a nucleotide sequence wherein 1 to 10 (for example, 1 to 5, preferably 1 to 3, more preferably 1 to 2) nucleotide base(s) is/are substituted with other nucleotide base(s) in the nucleotide sequence of SEQ ID No: 2 or 5, (iii) a nucleotide sequence wherein 1 to 10 (for example, 1 to 5, preferably 1 to 3, more preferably 1 to 2) nucleotide base(s) is/are added in the nucleotide sequence of SEQ ID No: 2 or 5, or (iv) a nucleotide sequence of a combination of the above (i) to (iii).

A polynucleotide comprising a nucleotide sequence in which one or a plurality of amino acids have been deleted, substituted or added in the nucleotide sequence of SEQ ID NO: 2 or 5 can be prepared, for example, according to site-directed mutagenesis described in “Molecular Cloning, A Laboratory Manual 2nd ed.” (Cold Spring Harbor Press (1989)), “Current Protocols in Molecular Biology” (John Wiley & Sons (1987-1997)), Kunkel (1985) Proc. Natl. Acad. Sci. USA 82: 488-92, Kunkel (1988) Method. Enzymol. 85: 2763-6 and others.

In order to introduce mutation into a polynucleotide, it can be carried out according to a known method such as Kunkel method and Gapped duplex method by using a mutagenesis Kit for site-directed mutagenesis such as QuikChange™ Site-Directed Mutagenesis Kit (Stratagene Corp.) GeneTailor™ Site-Directed Mutagenesis System (Invitrogen. Corp.), or TaKaRa Site-Directed Mutagenesis System (Takara Bio Inc.: Mutan-K, Mutan-Super Express Km and the like).

The present polynucleotide (C) can be prepared, for example, as follows.

A cDNA library can be prepared according to a conventional genetic engineering technique (for example, a method described in “Shin Saibokogaku Jikken Protocol” (edited by Tokyo University, Medical Science Laboratoty, Oncology Research Department; Shunjunsha, 1993) from fresh horse liver and others, and PCR can be performed by using the prepared cDNA library as a temperate and using appropriate primers, thereby amplifying DNA comprising a nucleotide sequence encoding the amino acid sequence of SEQ ID NO: 1; DNA comprising a nucleotide sequence encoding an amino acid sequence in which one or a plurality of amino acids have been deleted, substituted or added in the amino acid sequence of SEQ ID NO: 1 and/or DNA having the nucleotide sequence of SEQ ID NO: 2, and others, to prepare DNA of the present polynucleotide.

PCR can be performed by using, as a template, the above cDNA library and using, as primers, an oligonucleotide having the nucleotide sequence of SEQ ID NO: 3 and an oligonucleotide having the nucleotide sequence of SEQ ID NO: 4, thereby amplifying DNA comprising the nucleotide sequence of SEQ ID NO: 2, to prepare DNA of the present polynucleotide.

Examples of the condition of the PCR include a condition in which a reaction solution obtained by mixing 4 kinds of dNTPs each in amount of 20 μM, 2 kinds of oligonucleotide primers each in amount of 15 μmol, Taq polymerase in an amount of 1.3 U and a cDNA library as a template is heated at 97° C. (for 2 minutes), then, a cycle of 97° C. (for 0.25 minutes)-50° C. (for 0.5 minutes)-72° C. (for 1.5 minutes) is repeated 10 times, then, a cycle of 97° C. (for 0.25 minutes)-55° C. (for 0.5 minutes)-72° C. (for 2.5 minutes) is repeated 20 times, further, the reaction solution is kept at 72° C. for 7 minutes.

A restriction enzyme recognition sequence or the like may be added to the 5′ end of a primer used for the PCR.

It is also possible to perform PCR using, as a template, the above-mentioned cDNA library and using, as a primer, an oligonucleotide having a partial nucleotide sequence selected from among nucleotide sequences coding an amino acid sequence of SEQ ID NO: 1 (for example, oligonucleotide comprising a nucleotide sequence of about nucleotide bases or more at the 5′ end side of a nucleotide sequence coding the amino acid sequence of SEQ ID NO: 1) and an oligonucleotide of about 14 nucleotide bases or more comprising a nucleotide sequence complementary to a nucleotide sequence near a DNA insertion site of a vector used for generating the cDNA library, thereby amplifying DNA having a nucleotide sequence encoding the amino acid sequence of SEQ ID NO: 1, DNA comprising a nucleotide sequence encoding an amino acid sequence in which one or a plurality of amino acids have been deleted, substituted or added in the amino acid sequence of SEQ ID NO: 1, and the like, to prepare DNA of the present polynucleotide.

DNA amplified as described above can be cloned to a vector according to a method described in “Molecular Cloning: A Laboratory Manual 2nd edition” (1989), Cold Spring Harbor Laboratory Press, “Current Protocols in Molecular Biology” (1987), John Wiley & Sons, Inc. ISBNO-471-50338-X, and the like, to obtain a recombinant vector. Specific examples of the vector to be used include pUC119 (Takara Shuzo Co., Ltd.), pTV118N (Takara Shuzo Co., Ltd.), pBluescriptII (Toyobo Co., Ltd.), pCR2.1-TOPO (Invitrogen), pTrc99A (Pharmacia), pKK223-3 (Pharmacia) and the like.

DNA of the present polynucleotide can also be obtained by hybridizing, under conditions described below, as a probe, DNA of about 15 nucleotide bases or more having a partial nucleotide sequence selected from among nucleotide sequences coding an amino acid sequence of SEQ ID NO: 1, with a cDNA library which has been inserted into a microorganism- or phage-derived vector, and detecting DNA to which the probe binds specifically.

Examples of the method of hybridizing a probe to chromosomal DNA or cDNA include colony hybridization and plaque hybridization, and the method can be selected depending on the kind of a vector used for preparing a library.

When a library to be used is prepared using a plasmid vector, it is recommendable to use colony hybridization. Specifically, DNA of a library is introduced into a host microorganism to obtain transformants, the resulting transformants are diluted, then, the diluted product is inoculated on an agar medium, and culturing is performed until appearance of a colony.

When a library to be used is prepared using a phage vector, it is recommendable to use plaque hybridization. Specifically, a host microorganism and a phage of a library are mixed under infectable condition, further mixed with a soft agar medium, then, the mixture is inoculated on an agar medium, and culturing is performed until appearance of a plaque.

Then, in any hybridization cases, a membrane is placed on the agar medium on which the above-mentioned culturing has been effected, and a transformant or phage is adsorbed and transferred to the membrane. This membrane is treated with an alkali, then, neutralized, then, DNA is fixed to the membrane. As more specific examples of plaque hybridization, a nitrocellulose membrane or nylon membrane (for example, Hybond-N⁺ (registered trademark of Amersham)) is placed on the above-mentioned agar medium, and allowed to stand still for about 1 minute to cause adsorption and transfer of phage particles to a membrane. Then, the membrane is immersed in an alkali solution (for example, 1.5 M sodium chloride, 0.5 M sodium hydroxide) for about 3 minutes to cause dissolution of phage particles, thereby, eluting phage DNA on a membrane, then, immersed in a neutralization solution (for example, 1.5 M sodium chloride, 0.5 M Tris-HCl buffer, pH 7.5) for about 5 minutes. Then, the membrane is washed with a washing solution (for example, 0.3 M sodium chloride, 30 mM citric acid, 0.2 M Tris-HCl buffer, pH 7.5) for about 5 minutes, then, for example, heated at about 80° C. for about 90 minutes, to fix phage DNA on the membrane.

Using thus prepared membrane, hybridization is carried out using the above-mentioned DNA as a probe. Hybridization can be conducted, for example, according to descriptions of J. Sambrooke, E. F. Frisch, T. Maniatis, “Molecular Cloning: A Laboratory Manual 2nd edition (1989)”, Cold Spring Harbor Laboratory Press, and the like.

DNA used as a probe may be one labeled with a radioisotope, or one labeled with a fluorescent coloring matter.

As the method of labeling DNA used as a probe with a radioisotope, there is, for example, a method of performing PCR using, as a template, DNA used as a probe, replacing dCTP in the PCR reaction solution with (α-32P)dCTP, by utilizing Random Primer Labeling Kit (Takara Shuzo Co., Ltd.) and the like.

When DNA used as a probe is labeled with a fluorescent coloring matter, there can be used, for example, ECL Direct Nucleic Acid Labeling and Detection System manufactured by Amersham, and the like.

Hybridization can be performed, for example, as described below.

A prehybridization solution containing 450 to 900 mM sodium chloride and 45 to 90 mM sodium citrate, containing sodium dodecylsulfate (SDS) in a concentration of 0.1 to 1.0 wt %, containing denatured non-specific DNA in a concentration of 0 to 200 μl/ml, and depending on conditions, optionally containing albumin, ficoll, polyvinylpyrrolidone and the like each in a concentration of 0 to 0.2 wt % (preferably, prehybridization solution containing 900 mM sodium chloride, 90 mM sodium citrate, 1.0 wt % SDS and 100 μl/ml denatured Calf-thymus DNA) is prepared in a proportion of 50 to 200 μl per 1 cm² of a membrane produced as described above, and the above-mentioned membrane is immersed in the prehybridization solution and kept at 42 to 65° C. for 1 to 4 hours.

Next, for example, a prehybridization solution containing 450 to 900 mM sodium chloride and 45 to 90 mM sodium citrate, containing SDS in a concentration of 0.1 to 1.0 wt %, containing denatured non-specific DNA in a concentration of 0 to 200 μl/ml, and depending on conditions, optionally containing albumin, ficoll, polyvinylpyrrolidone and the like each in a concentration of 0 to 0.2 wt % (preferably, prehybridization solution containing 900 mM sodium chloride, 90 mM sodium citrate, 1.0 wt % SDS and 100 μg/ml denatured Calf-thymus DNA) is mixed with a probe prepared by the above-mentioned method (amount corresponding to 1.0×10⁴ to 2.0×10⁶ cpm per 1 cm² of membrane) to give a solution which is prepared in a proportion of 50 to 200 μl per 1 cm² of the membrane, and the membrane is immersed in the hybridization solution and kept at 42 to 65° C. for 12 to 20 hours.

After the hybridization, the membrane is taken out, and washed using a washing solution of 42 to 65° C. containing 15 to 300 mM sodium chloride, 1.5 to 30 mM sodium citrate and 0.1 to 1.0 wt % SDS and the like (preferably, washing solution of 65° C. containing 15 mM sodium chloride, 1.5 mM sodium citrate and 1.0 wt % SDS). The washed membrane is rinsed slightly with 2×SSC (300 mM sodium chloride, 30 mM sodium citrate), then, dried. This membrane is subjected to, for example, autoradiography and the like to detect a position of a probe on the membrane, thereby specifying, on the original agar medium, a clone that is hybridized with a probe used and corresponds to a position of DNA on the membrane, and this is picked up to isolate a clone having the DNA.

The present polynucleotide can be prepared from a cultured microbial cell obtained by culturing thus obtained clone.

The present polynucleotide can be synthesized artificially. A design and synthesis of an artificial-synthetic gene can be carried out by referring to a method described in Cell Technology additional volume, plant cell technology series 7, “PCR experimental protocol of plant”, page 95-100, Takumi Shimamoto and Takuji Sasaki supervised, Shujunsha published, issued on Jul. 1, 1997.

Specifically, for example, a nucleotide sequence of the present polynucleotide is designed by creating the amino acid sequence of SEQ ID NO: 1 and selecting, as a codon corresponding to each amino acid included in the amino acid sequence, a codon that is frequently used in a microorganism cell (for example, E. Coli.) which will be used to express the codon. The information on codon usage in E. Coli and others is, for example, available from a DNA data base well-known to those skilled in the art (GenBank, EMBL, DDBJ and others). Also when Bacillus subtilis is the microorganism cell and a signal sequence is added to the above amino acid sequence to transport it extracellularly, an amino acid sequence of the whole protein including the signal sequence is created. The signal sequence is preferably one derived from the microorganism cell, and examples thereof may include a signal sequence of α-amylase of Bacillus subtilis and others, which is an extracellular transfer signal.

Hereinafter, specific experimental procedures are described.

First, the number of each amino acid included in the created amino acid sequence is calculated. To become the nearest an average appearance frequency of the codon of a microorganism cell that will be used to express the polynucleotide, codons for amino acids of the number calculated above are allocated. The order of using each codon is applied so that the same codon will be not consecutive as much as possible. It is selected sequentially from amino acids on N-terminal side in the order of the codons decided on each amino acid, then the codon of the each amino-acid residue is temporarily decided. The codons of all amino acids to the C-terminus are temporarily decided by repeating these procedures, and finally the termination codon is arranged. On the artificial gene composed of the decided codons, it is checked that neither the nucleotide sequence that inhibits the transcription of the gene in the microorganism cell nor the nucleotide sequence that the restriction enzyme used in the operation described below recognizes exists. When such a nucleotide sequence exists, the codon that is allocated in this nucleotide sequence is exchanged for a codon used in another part. When the gene is designed, it is preferable to add nucleotide sequences which are recognized by restriction enzymes suitable for the operation described below to 5′-end and 3′-end of the gene.

Next, the gene having the nucleotide sequence designed above can be synthesized using long length chain DNA synthesis method that uses PCR (Shimamoto, et al., “PCR experiment protocol of plant”, refer to the above) (hereinafter, this method may be sometimes referred to as “Assembly PCR method”). In the method, DNA is synthesized using long synthetic oligonucleotide primers only. The pair of the primer is synthesized so that the complementary strand or the overlap of about 10 to 12 bp will be present at each 3′-end, and primers each other are used as a template, then, DNA is synthesized. The total length of the primer may be, for example, about 60 to 100 mer. Preferably, it may be, for example, about 80 to 100 mer.

First, based on the designed nucleotide sequence, for example, DNA oligomers that made a primer every about 90 nucleotide bases are designed and synthesized. The synthesis of DNA oligomer can be carried out with DNA synthesizer by β-cyanoethylphosphoramidite method. For example, a first DNA oligomer is designed and synthesized using the designed nucleotide sequence from the vicinity of the center part to about 90 residues upstream on the 5′-side. Next, a complementary strand oligomer, that contains a nucleotide sequence of 12 residues on the 3′-side of the first DNA oligomer and has a long of about 90 residues downstream on the 3′-side from this part, are synthesized and is defined as the second DNA oligomer. Also, a complementary strand oligomer, that contains a nucleotide sequence of 12 residues on the 5′-side of the first DNA oligomer and has a long of about 90 residues upstream on the 5′-side from this part, are synthesized and is defined as the third DNA oligomer. Further, a complementary strand oligomer, that contains a nucleotide sequence of 12 residues on the 5′-side (the 3′-site from the view of the gene site) of the second DNA oligomer and has a long of about 90 residues upstream on the 3′-side from this part, are synthesized and is defined as the firth DNA oligomer. Subsequently, in the same way, an appropriate amount of DNA oligomers are synthesized. When desired polynucleotide has not been covered, the oligomers are further synthesized until it is covered.

Next, these oligomers are sequentially bound by the PCR reaction. First, PCR reaction is carried out using the first DNA oligomer and the second DNA oligomer as primers. PCR reaction is carried out by making the obtained PCR products a template and using the third DNA oligomer and the fourth DNA oligomer as primers.

The PCR reaction is, for example, 5 cycles of one set of denaturation temperature 94° C. (for 1 minutes), anneal temperature 51° C. (1 minutes), extension temperature 72° C. (for 2 minutes) and thereafter, 20 cycles of one set of denaturation temperature 94° C. (1 minutes), anneal temperature 60° C. (1 minutes), extension temperature 72° C. (2 minutes). It is preferable that DNA polymerase used in the reaction uses an enzyme with low incorporating error rate of the nucleotide base. Subsequently, the nucleotide sequence is extended by repeating the above operation, to obtain a desired nucleotide sequence. The restriction enzyme site is installed at both ends of a desired nucleotide sequence as needed, and it introduces into a cloning vector according to a common procedure, then a subcloning is done. The nucleotide sequence of the obtained clone is confirmed with DNA sequencer, and thus it is confirmed to have obtained a polynucleotide having a desired nucleotide sequence. Thus, for example, DNA of the present polynucleotide can be prepared by artificially synthesizing DNA having the nucleotide sequence set forth in SEQ ID NO: 5.

For allowing the present polynucleotide thus obtained to express in a microorganism cell, for example, a DNA in which a promoter functional in the microorganism cell is operably linked to the present polynucleotide is introduced into the microorganism cell.

Here, “operably linked” means that when a microorganism cell is transformed by introducing the above DNA into the microorganism cell, the present polynucleotide is under condition of bonding to a promoter so as to be expressed under control of the promoter. Examples of the promoter includes a lac promoter of lactose operon of E. coli, a trp promoter of tryptophan operon of E. coli, or synthetic promoters that are uniquely-altered and designed to be functionable in E. coli such as tac promoter, trc promoter and others. Also PL promoter or PR promoter of Lambda Phage origin, gluconate synthase promoter of Bacillus subtilis origin (gnt), alkaline protease promoter (apr), neutral protease promoter (npr), and α-amylase promoter (amy) and others are included.

In general, DNA operably linked to a promoter functional in a microorganism cell can be cloned to a vector according to a method described in “Molecular Cloning: A Laboratory Manual 2nd edition” (1989), Cold Spring Harbor Laboratory Press, “Current Protocols in Molecular Biology” (1987), John Wiley & Sons, Inc. ISBNO-471-50338-X, to obtain a recombinant vector.

The vector to be used should not be limited as long as it can maintain the present polynucleotide and is replicable (for example, a vector containing DNA sequence, promoter, ribosome binding sequence, transcription terminator (transcription termination sequence), selective marker gene, which is necessary for plasmid to grow in microbial cell), and the vector that is appropriate for each host can be used. For example, plasmid DNA and bacteriophage and others can be included.

Examples of plasmid DNA include plasmid of E. coli. origin (ColE plasmid such as pBR322, pUC18, pUC19, pUC118, pUC119 (TAKARA SHUZO CO., LTD.), pTV118N (TAKARA SHUZO CO., LTD.), pBluescriptII (TOYOBO), pCR2.1-TOPO (Invitrogen), pTrc99A (Pharmacia), pKK223-3 (Pharmacia) and others), plasmid of Actinomycetes origin (pIJ486 and others), plasmid of Yeast origin (YEp13, YEp 24, Ycp50 and others). Examples of phage DNA includes λpharge (Charon4A, Charon21A, EMBL3, EMBL4, λgt10, λgt11 and others), retrotransposon DNA, artificial chromosome and others.

Also as to a vector, when a vector containing a selective marker gene (antibiotic resistance-imparted gene such as, dehydrofolate reductase gene, kanamycin-resistant gene, ampicillin-resistant gene, neomycin-resistant gene, blasticidin-resistant gene and others) is used as the vector, a transformant containing the vector introduced can be selected utilizing the phenotype of the selective marker gene and the like as an index. Also, the SD sequence and the Kozak sequence are known as a ribosome binding sequence, and these sequences can be inserted into the upstream of the mutation gene. Utilizing the PCR method and others, the SD sequence may be added when prokaryote is used as a host and the Kozak sequence may be added when eukaryote is used as a host. Examples of the SD sequence include a sequence of E. coli. origin, Rhodococcus bacteria origin, or Bacillus subtilis origin and others, but should not be limited as long as the nucleotide sequence is one that can function in the desired microbial cell. For example, consensus sequence in which a nucleotide sequence complementary to 3′ terminal region of 16S ribosome RNA is consecutive more than four nucleotide bases is produced by the DNA synthesis method, then may be utilized. Though transcription termination sequence is not necessarily necessary, ρ factor independent one such as lipoprotein terminator and trp operon terminator and others may be utilized.

An incorporation of the present polynucleotide into such a vector can be carried out by cutting DNA containing the present polynucleotide with an appropriate restriction enzyme and if necessary, adding an appropriate linker to the DNA and thereafter, by binding the DNA to the vector that is cut with an appropriate restriction enzyme. Also another method can be carried out by subjecting the DNA containing the present polynucleotide to PCR amplification using a primer containing an appropriate restriction enzyme recognition site, then, processing the amplified product with the restriction enzyme and thereafter, by binding the DNA to the vector that is cut with an appropriate restriction enzyme.

If the recombinant vector thus produced is introduced in the microorganism cell, the transformant that expresses the present enzyme highly can be obtained. It is possible to express the present enzyme by culturing the transformant.

The method of introducing the present polynucleotide operably linked to a promoter functional in a microorganism cell or the recombinant vector retaining the polynucleotide, into a microorganism cell, may be a DNA introduction method conventionally used depending on the microorganism cell to be used, and examples thereof include a calcium chloride method described in “Molecular Cloning: A Laboratory Manual 2nd edition” (1989), Cold Spring Harbor Laboratory Press, “Current Protocols in Molecular Biology” (1987), John Wiley & Sons, Inc. ISBNO-471-50338-X, and the like, a Heat-Shock method, a Spheroplast method, a lithium acetate method, or an electroporation method described in “Methods in Electroporation: Gene Pulser/E. coli Pulser System” Bio-Rad Laboratories (1993) and others.

Examples of “microorganism cell” herein include microorganisms such as E. coli (specifically, for example, K12 strain, B strain, JM109 strain, XL1-Blue strain, C600 strain, w3110 strain), Bacillus subtilis, yeast, fungus, Rhodococcus genus. Preferably, microorganisms belonging to Escherichia genus, Bacillus genus, Corynebacterium genus, Staphylococcus genus, Streptomyces genus, Saccharomyces genus (specifically, for example, Saccharomyces cerevisiae), Schizosaccharomyces genus (specifically, for example, Schizosaccharomyces pombe), Pichia genus (specifically, for example, Pichia pastoris), Kluyveromyces genus, Aspergillus genus and Rhodococcus genus (specifically, for example, Rhodococcus rhodochrous ATCC 12674 strain, Rhodococcus rhodochrous J-1 strain (FERM BP-1478)) and others are included.

For selecting the transformant into which the present polynucleotide operably linked to a promoter functional in a microorganism cell or the recombinant vector retaining the polynucleotide and others has been introduced, for example, it is recommendable to select the transformant utilizing the phenotype of a selective marker gene contained in a vector as described above as an indicator.

A fact that the transformant obtained retains the present polynucleotide can be checked, for example, by performing recognition of a restriction enzyme site, analysis of a nucleotide sequence, Southern Hybridization, Western Hybridization and the like, according to usual methods described in “Molecular Cloning: A Laboratory Manual 2nd edition” (1989), Cold Spring Harbor Laboratory Press, and others.

For example, a microbial cell of the present transformant is reacted with a sulfur-containing amino alcohol compound. Then a fact that the obtained DNA encodes an amino acid sequence of an enzyme which has the above-mentioned abilities can be checked by analyzing an amount of the sulfur-containing α-amino aldehyde compound and/or the sulfur-containing α-amino acid compound in a reaction product.

Similarly, the fact can be checked by sequencing a nucleotide sequence of DNA using a conventional method. For example, sequencing is carried out by Dideoxy Chain Termination Method (see, for example, F. Sanger, S, Nicklen, A. R. Coulso, Proceeding of Natural Academy of Science U.S.A. (1977) 74: pages 5463-5467). The sample preparation for a nucleotide sequence analysis may be used commercial reagents such as ABI PRISM Dye Terminator Cycle Sequencing Ready Reaction Kit (Perkin-Elmer Corp.). Also the nucleotide sequence can be analyzed utilizing an appropriate DNA sequencer.

Hereinafter, a method for the preparation of the present transformant by a culturing method is explained.

The present transformant may be cultured in a culture medium for culturing various microorganisms which appropriately contain a carbon source, a nitrogen source, an organic salt, an inorganic salt, and others.

Examples of the carbon source include sugars such as glucose, dextrin and sucrose; sugar alcohols such as glycerol; organic acids such as fumaric acid, citric acid and pyruvic acid; animal oils; vegetable oils; and molasses. These carbon sources are added to the culture medium in an amount of usually about 0.1% (w/v) to 30% (w/v) of the culture.

Examples of the nitrogen source include natural organic nitrogen sources such as meat extract, peptone, yeast extract, malt extract, soy flour, Corn Steep Liquor, cottonseed flour, dried yeast and casamino acid; amino acids; sodium salts of inorganic acids such as sodium nitrate; ammonium salts of inorganic acids such as ammonium chloride, ammonium sulfate and ammonium phosphate; ammonium salts of organic acids such as ammonium fumarate and ammonium citrate; and urea. Among these nitrogen sources, ammonium salts of organic acids, natural organic nitrogen sources, and amino acids and others may also be used as carbon sources in many cases. The above nitrogen sources are added to the culture medium in an amount of usually about 0.1% (w/v) to 30% (w/v) of the culture.

Examples of the organic salt and inorganic salt include chloride, sulfate, acetate, carbonate and phosphate of potassium, sodium, magnesium, iron, manganese, cobalt, zinc, and others. Specific examples thereof include sodium chloride, potassium chloride, magnesium sulfate, ferrous sulfate, manganese sulfate, cobalt chloride, zinc sulfate, copper sulfate, sodium acetate, calcium carbonate, potassium hydrogen phosphate and dipotassium hydrogen phosphate. These organic salts and/or inorganic salts are added to the culture medium in an amount of usually about 0.0001% (w/v) to 5% (w/v) of the culture.

In culturing a transformant in which has been introduced DNA in which the present polynucleotide is operably linked to an inducible promoter as a promoter, an inducer may be added into culture medium as needed. For example, in culturing a transformant that is introduced with DNA in which the present polynucleotide is operably linked to an allolactose-induced type promoter such as tac promoter, trc promoter and lac promoter, an inducible agent for inducing a production of the present enzyme such as isopropyl thio-β-D-galactoside (IPTG) can be added in a small amount into culture medium. Also, in culturing a transformant that is introduced with DNA in which the present polynucleotide is operably linked to an indoleacetic acid (IAA)-induced type promoter such as trp promoter, an inducible agent for inducing a production of the present enzyme such as IAA can be added in a small amount into culture medium.

Examples of the culture method include solid culture and liquid culture (e.g. a test tube culture, a flask culture, or a jar fermenter culture).

Culture temperature and pH of the culture are not particularly limited as long as the present transformants are able to grow in the range thereof. For example, the culture temperature may be in a range of about 15° C. to about 45° C., preferably between 10° C. and about 37° C. and the pH of the culture may be in a range of about 4 to about 8. The culture time may be appropriately selected depending on the culture conditions, and is usually about 1 day to about 7 days.

The cultured product of the present transformants can be directly used as a catalyst for the process of the present invention. Examples of a method for using the cultured product of the present transformant include the following (1) and (2):

(1) a method for directly using culture, and

(2) a method for using microbial cells collected by centrifuging a culture (wet microbial cells washed as needed with buffer water).

The processed products of the cultured product of the present transformants may also be used as a catalyst for the process of the present invention. Examples of the processed products of the microbial cells include microbial cells obtained by culturing followed by treating with an organic solvent (e.g. acetone and ethanol), lyophilizing, or treating with alkali; the physically or enzymatically disrupted microbial cells; and crude enzymes separated or extracted from the these microbial cells. Furthermore, examples of the processed products include those immobilized by a known method after the above-mentioned treatments.

As the method of purifying the present enzyme from a cultured product of the present transformant, methods used in usual protein purification can be applied, and for example, the following methods are mentioned.

First, microbial cells are collected from a cultured product of the present transformant by centrifugal separation and the like, thereafter, these are disrupted by physical crushing methods such as ultrasonic treatment, dynomill treatment, French press treatment and the like or chemical crushing methods using a surfactant or a bacteriolysis enzyme such as lysozyme and the like. Impurities are removed from the resulting disrupted liquid by centrifugal separation, membrane filter filtration and the like to prepare cell-free extract, and this is fractioned appropriately using separation and purification methods such as cation exchange chromatography, anion exchange chromatography, hydrophobic chromatography, gel filtration chromatography, metal chelate chromatography and the like, thus, the present enzyme can be purified.

As the carrier to be used in chromatography, for example, insoluble polymer carriers such as cellulose, dextrin and agarose containing a carboxymethyl (CM) group, diethylaminoethyl (DEAE) group, phenyl group or butyl group introduced, and the like is included. Commercially available carrier-filled columns can also be used, and examples of such commercially available carrier-filled columns include Q-Sepharose FF, Phenyl-Sepharose HP ((registered trade mark) which are both manufactured by Amersham Pharmacia Biotech), TSK-gel G3000SW ((registered trade mark) Toso Co., Ltd.), and the like.

For selecting a fraction containing the present enzyme, it is recommendable to select a fraction depending on existence or nonexistence or a degree of “capability of converting a sulfur-containing amino alcohol compound into a corresponding sulfur-containing α-amino aldehyde compound”, “capability of converting a sulfur-containing α-amino aldehyde compound into a corresponding sulfur-containing α-amino acid compound”, and/or “both of capability of converting a sulfur-containing amino alcohol compound into a corresponding sulfur-containing α-amino aldehyde compound and capability of converting a sulfur-containing α-amino aldehyde compound into a corresponding sulfur-containing α-amino acid compound” in the present invention.

Specific embodiments include the microbial cells of the present transformants and the processed products thereof (e.g. cell-free extracts, partially purified proteins, purified proteins and immobilized materials thereof). Examples of the processed products of the cultured products include lyophilized microorganisms, organic solvent-treated microorganisms, dried microorganisms, disrupted microorganisms, autolysates of microorganisms, sonicated microorganisms, extracts of microorganisms, and alkali-treated microorganisms. Furthermore, examples of a method of obtaining the immobilized materials include carrier binding methods (e.g. a method of adsorbing proteins and others on to inorganic carriers such as silica gel and ceramics, cellulose, or ion-exchanged resin) and encapsulating methods [e.g. a method of trapping proteins and others in a network structure of macromolecules such as polyacrylamide, sulfur-containing polysaccharide gel (e.g. carrageenan gel), alginate gel, and agar gel].

In the event that the present transformants are used in the industrial production process, the products of killed microorganisms might be preferred to unprocessed microorganisms from the point of view of limitation of manufacturing equipments or other factors. Examples of a method for killing the microorganisms include physical sterilization (e.g. heating, drying, freezing, irradiation, sonication, filtration, and electric sterilization) and sterilization with chemical agents (e.g. alkalis, acids, halogens, oxidizing agents, sulfur, boron, arsenic, metals, alcohols, phenols, amines, sulfides, ethers, aldehydes, ketones, cyan, and antibiotics). Among these killing methods, generally, it is preferable to select a method which can lower the amount of residues or contaminants in the reaction system and can minimize inactivation of the above-described “capability of converting a sulfur-containing amino alcohol compound into a corresponding sulfur-containing α-amino aldehyde compound”, “capability of converting a sulfur-containing α-amino aldehyde compound into a corresponding sulfur-containing α-amino acid compound”, and/or “both capability of converting a sulfur-containing amino alcohol compound into a corresponding sulfur-containing α-amino aldehyde compound and capability of converting a sulfur-containing α-amino aldehyde compound into a corresponding sulfur-containing α-amino acid compound”.

Hereinafter, the present invention is explained more specifically.

### 1. A Processed Product of Cultured Products

### First Embodiment

The centrifuge separation method and the film filtration method can be used to collect the microbial cell from the culture products of the present transformant. While the condition of the centrifuge separation should not be limited, the method can be carried out, for example, under the condition of 3,000 to 4,500×g at 4° C. for 5 to 20 minutes. The collected present transformant can be, as needed, washed with monosodium phosphate buffer, phosphate buffer and others, then suspended. The suspension of the microbial cell is thus obtained.

As a method of disrupting microbial cell, ultrasonic treatment, high-pressure treatment with French press treatment or homogenizer, grinding treatment with glass beads, enzymatic treatment with lysozyme, cellulase and pectinase, freeze-thaw treatment, treatment with hypotonic fluid, lytic induction treatment with phage and others can be utilized. The disruption treatment is carried out under ice-cooling as needed. For example, the suspension of the microbial cell may be disrupted under ice-cooling for 1 to 5 minutes, preferably for 3 minutes with ultrasonic vibrator VP-15S (Taitec, Japan) in the setting condition of Output control 4, DUTY CYCLE 40%, PULS, TIMER=B mode 10 s. Also, for example, the suspension of the microbial cell may be disrupted under 100 MPa pressurized condition with a homogenizer PANDA2K type manufactured by Niro Soavi.

After crushing, the disrupted residue of the microbial cell can be removed from the disrupted products of the present transformant as needed. Examples of the method of removing the residue include centrifuge separation method and filtration and others. As needed, residue removal efficiency can be raised using an aggregating agent or filter aid and others. Though the condition of the centrifuge separation should not be limited, the method can be carried out, for example, under the condition of 4,000 to 25,000×g at 4° C. for 3 to 45 minutes. The residue may be thus removed from the disrupted products.

### 2. A Processed Product of Cultured Products

### Second Embodiment

A lot of proteins other than the present enzyme can be denatured by heating-treatment of the above-mentioned disrupted products of the present transformant or cell-free extracts. Thus, the solution of the present enzyme can be obtained as a soluble fraction by heating-treatment of the disrupted products of the present transformant or cell-free extracts. The present enzyme includes the solution of the present enzyme obtained as described above.

“Heating-treatment” herein referred to as a thermal deactivation procedure to denature the protein other than the present enzyme derived from the present transformant and a temperature of heating-treatment is preferably between 50° C. or more and 75° C. or less, more preferably between 60° C. or more and 70° C. or less. Though the period of heating-treatment should not be limited, it is preferable to be 10 minutes or more after the disrupted products of the present transformant or the cell-free extracts reaches at a preset temperature. More preferred period is between 1 hour or more and 5 hours or less.

For example, the heating-treatment can be carried out by placing the disrupted products of the present transformant and others in a test tube, and then incubating the test tube in water bath that set to a given temperature for a given period. Also the heating-treatment can be carried out by placing the disrupted products of the present transformant and others in three-neck flask with a thermometer, and then heating the flask until the given temperature for a given period.

In the present invention, it may be carried out by heat-treating the disrupted products of the present transformant (i.e. preheating), and then removing the disrupted residue and thereafter, heat-treating it again. At reheating, zinc salt may be existed.

Examples of the method of removing an insoluble material formed by heat-treating include centrifuge separation method and filtration and others, and as needed, residue removal efficiency can be raised using an aggregating agent or filter aid and others. If necessary, it may be further purified using various chromatographies (gel filtration, ion-exchange chromatography, affinity chromatography and others).

The process of the present invention is usually carried out in the presence of water, preferably, in an additive system with coenzyme such as oxidized nicotinamide adenine dinucleotide (hereinafter, referred to as NAD⁺). The water used in this case may be in the form of a buffer. Examples of buffer agents used in the buffer include alkali metal salts of phosphoric acid such as sodium phosphate and potassium phosphate, and alkali metal salts of acetic acid such as sodium acetate and potassium acetate. Examples of alkaline buffer include Tris-HCl buffer, Tris-citrate buffer, and Tris-glycine buffer.

The process of the present invention may also be carried out by additionally using a hydrophobic organic solvent, i.e. in the presence of water and the hydrophobic organic solvent. Examples of the hydrophobic organic solvent used in this case include esters such as ethyl formate, ethyl acetate, propyl acetate, butyl acetate, ethyl propionate and butyl propionate, alcohols such as n-butyl alcohol, n-amyl alcohol and n-octyl alcohol, aromatic hydrocarbons such as benzene, toluene and xylene, ethers such as diethylether, diisopropylether and methyl-t-butylether, halogenated hydrocarbons such as chloroform and 1,2-dichloroethane, and mixtures thereof.

The process of the present invention may also be carried out by additionally using a hydrophilic organic solvent, i.e. in the presence of water and an aqueous medium. Examples of the hydrophilic organic solvent used in this case include alcohols such as methanol and ethanol, ketones such as acetone, ethers such as dimethoxyethane, tetrahydrofuran and dioxane, dimethylsulfoxide, and mixtures thereof.

While the process of the present invention is usually carried out in a range of pH of aqueous layer of 3 to 11, the pH may be appropriately changed in such a range that the reaction proceeds. It is preferable that the process of the present invention be carried out in the alkaline range, and it is more preferable that the process be carried out in a range of pH of aqueous layer 8 to 10.

While the process of the present invention is usually carried out in a range of about 0° C. to about 60° C., but the temperature may be appropriately changed in such a range that the reaction proceeds.

The process of the present invention is usually carried out in a range of for about 0.5 hours to about 10 days. After the completion of adding the sulfur-containing amino alcohol compound represented by the formula (1) [i.e. Compound (1)], which is the starting compound, the endpoint of the reaction can be checked, for example, by measuring the amount of the sulfur-containing amino alcohol compound of the formula (1) in the reaction solution by liquid chromatography or gas chromatography and the others.

The concentration of the sulfur-containing amino alcohol compound represented by the formula (1) [i.e. Compound (1)], which is the starting compound used in the process of the present invention, is usually 50% (w/v) or less and the sulfur-containing amino alcohol compound of the formula (1) [i.e. Compound (1)] may be continuously or successively added to a reaction system in order to maintain the concentration of the sulfur-containing amino alcohol compound of the formula (1) in the reaction system nearly constant.

During the process of the present invention, for example, a sugar such as glucose, sucrose or fructose, or a surfactant such as Triton X-100 (registered trade mark) or Tween 60 (registered trade mark) may be added to the reaction system if necessary.

In the process described in the specification, as aforementioned, the process of the present invention and the enzymatic method of the present invention are usually carried out in the presence of water, preferably, in a system added by coenzyme such as oxidized nicotinamide adenine dinucleotide (hereinafter, sometimes referred to as NAD⁺). In the above additive system, the NAD⁺ is converted into reduced (3-nicotinamide adenine dinucleotide (hereinafter, sometimes referred to as NADH) with progress of an oxidative reaction of a sulfur-containing amino alcohol compound or an oxidative reaction of a sulfur-containing α-amino aldehyde compound. The NADH generated by conversion can be returned into the original NAD⁺ by a protein capable of converting NADH into an oxidized form (NAD⁺), therefore, it is also possible to allow the protein capable of converting NADH into NAD⁺ to coexist in the reaction system of the above-mentioned method.

Specific example of the protein capable of converting reduced β-nicotinamide adenine dinucleotide (i.e. NADH) or NADPH into oxidized β-nicotinamide adenine dinucleotide (i.e. NAD⁺) or NADP⁺ includes lactate dehydrogenase and others.

When the protein capable of converting NADH or NADPH into NAD⁺ or NADP⁺ is a lactate dehydrogenase, the activity of the protein is reinforced in some cases by coexistence of sodium pyruvate and the like in the reaction system, and for example, these compounds may be added to the reaction solution.

The above protein may be an enzyme itself, or may coexist in the reaction system in the form of a microbial cell having the enzyme or a processed product of the microbial cell of the microorganism, or also may be a transformant or its processed product of the microbial cell containing polynucleotide having a nucleotide sequence coding an amino acid sequence of a protein capable of converting NADH or NADPH into NAD⁺ or NADP⁺. Such processed product of the microbial cell may be prepared according to the similar process to the aforementioned process of the processed product of the present transformant.

The process of the present invention can be utilized a transformant retaining simultaneously DNA having a nucleotide sequence coding an amino acid sequence of a protein capable of converting NADH or NADPH into NAD⁺ or NADP⁺ such as lactate dehydrogenase and others. Such transformant may be prepared according to the similar process to the above-mentioned process of the present transformant.

The collection of the sulfur-containing α-amino acid compound represented by the formula (2) from the reaction solution can be carried out by any methods known in the art.

For example, a typical method is a purification composed of post-treatments (e.g. organic solvent extraction from the reaction, concentration of the reaction, ion exchange method, and crystallization method), if necessary in combination with methods such as column chromatography and distillation.

The sulfur-containing amino acid compound represented by the formula (2) prepared by the process of the present invention may be in the form of a salt.

The present invention includes a process for producing a sulfur-containing α-amino acid compound represented by the formula (2) (i.e. the Compound (2)) comprising a step of reacting a sulfur-containing amino alcohol compound represented by the formula (1) (i.e. the Compound (1)) with an enzyme capable of converting the above sulfur-containing amino alcohol compound into a corresponding sulfur-containing α-amino aldehyde compound (i.e. the present enzyme (A)) (hereinafter, the above step will be sometimes referred as to ADH step) and a step of reacting the corresponding sulfur-containing α-amino aldehyde compound obtained in the above step with an enzyme capable of converting the above sulfur-containing α-amino aldehyde compound into a corresponding sulfur-containing α-amino acid compound (i.e. the present enzyme (A)) (hereinafter, the above step will be sometimes referred as to ADLH step) (hereinafter, the above method may be sometimes referred as to a first enzymatic method of the present invention). Also the present invention comprises a method for preparing a sulfur-containing α-amino acid compound represented by the formula (2) (i.e. the Compound (2)) comprising a step of reacting a sulfur-containing amino alcohol compound represented by the formula (1) (i.e. the Compound (1)) with an enzyme both capable of converting the above sulfur-containing amino alcohol compound into a corresponding sulfur-containing α-amino aldehyde compound and capable of converting the above sulfur-containing α-amino aldehyde compound into a corresponding sulfur-containing α-amino acid compound (i.e. the present enzyme (C)) (hereinafter, the above step may be sometimes referred as to ADH/ALDH step) (hereinafter, the above method may be sometimes referred as to a second enzymatic method of the present invention).

(hereinafter, the first enzymatic method of the present invention and the second enzymatic method of the present invention may be sometimes collectively referred to as an enzymatic method of the present invention)

The sulfur-containing amino alcohol compound used in the enzymatic of the present invention is, preferably, a sulfur-containing amino alcohol compound wherein R¹ is an alkyl group having 1 to 8 carbon atoms. Examples of more preferred compound include a sulfur-containing amino alcohol compound wherein R¹ is an alkyl group having 1 to 5 carbon atoms. Examples of further more preferred compound include a sulfur-containing amino alcohol compound wherein R¹ is a methyl group.

The enzymes used in the enzymatic method of the present invention, that is, an enzyme capable of converting the above sulfur-containing amino alcohol compound into a corresponding α-amino aldehyde compound (i.e. the present enzyme (A)), an enzyme capable of converting the above α-amino aldehyde compound obtained in the above step into a corresponding α-amino acid compound (i.e. the present enzyme (B)), and an enzyme both capable of converting the above sulfur-containing amino alcohol compound into a corresponding α-amino aldehyde compound and capable of converting the above α-amino aldehyde compound into a corresponding α-amino acid compound (i.e. the present enzyme (C)) are commercially available or may be obtained, for example, by purifying these enzymes from cultured products of the present transformants by applying a usual purification method of protein as above-mentioned. The more specific method for preparing these enzymes may be referred to the above-mentioned explanations or descriptions herein.

The ADH step in the first enzymatic method of the present invention is usually carried out in the presence of water, preferably, in an additive system with coenzyme such as oxidized nicotinamide adenine dinucleotide (hereinafter, referred to as NAD⁺). The water used in this case may be in the form of a buffer. Examples of buffering agents in the buffer include alkali metal salts of phosphoric acid such as sodium phosphate and potassium phosphate, alkali metal salts of acetic acid such as sodium acetate and potassium acetate. Examples of alkaline buffer Tris-HCl buffer, Tris-citrate buffer, and Tris-glycine buffer.

The ADH step in the first enzymatic method of the present invention may also be carried out by additionally using a hydrophobic organic solvent, i.e. in the presence of water and the hydrophobic organic solvent. Examples of the hydrophobic organic solvent used in this case include esters such as ethyl formate, ethyl acetate, propyl acetate, butyl acetate, ethyl propionate and butyl propionate, alcohols such as n-butyl alcohol, n-amyl alcohol and n-octyl alcohol, aromatic hydrocarbons such as benzene, toluene and xylene, ethers such as diethylether, diisopropylether and methyl-t-butylether, halogenated hydrocarbons such as chloroform and 1,2-dichloroethane, and mixtures thereof.

The ADH step in the first enzymatic method of the present invention may also be carried out by additionally using a hydrophilic organic solvent, i.e. in the presence of water and an aqueous medium. Examples of the hydrophilic organic solvent used in this case include alcohols such as methanol and ethanol, ketones such as acetone, ethers such as dimethoxyethane, tetrahydrofuran and dioxane, dimethylsulfoxide, and mixtures thereof.

While the ADH step in the first enzymatic method of the present invention is usually carried out in range of pH of aqueous layer of 3 to 11, the pH may be appropriately changed in such a range that the reaction proceeds. It is preferable that the process of the present invention be carried out in the alkaline range, and it is more preferable that the process be carried out in a range of pH of aqueous layer of 8 to 10.

While the ADH step in the first enzymatic method of the present invention is usually carried out in a range of about 0° C. to about 60° C., the temperature may be appropriately changed in such a range that the reaction proceeds.

The ADH step in the first enzymatic method of the present invention is usually carried out in a range of for about 0.5 hours to about 10 days. After the completion of adding the sulfur-containing amino alcohol compound represented by the formula (1) [i.e. Compound (1)], which is the starting compound, the endpoint of the reaction can be checked, foe example, by measuring the amount of the sulfur-containing amino alcohol compound of the formula (1) in the reaction solution by liquid chromatography or gas chromatography and the others.

The concentration of the sulfur-containing amino alcohol compound represented by the formula (1) [i.e. Compound (1)], which is the starting compound used in the first enzymatic method of the present invention, is usually 50% (w/v) or less and the sulfur-containing amino alcohol compound of the formula (1) [i.e. Compound (1)] may be continuously or successively added to a reaction system in order to maintain the concentration of the sulfur-containing amino alcohol compound of the formula (1) in the reaction system nearly constant.

During the ADH step in the first enzymatic method of the present invention, for example, a sugar such as glucose, sucrose or fructose, or a surfactant such as Triton X-100 (registered trade mark) or Tween 60 (registered trade mark)) may be added to the reaction system if necessary.

The ALDH step in the first enzymatic method of the present invention is usually carried out in the presence of water, preferably, in an additive system with coenzyme such as oxidized nicotinamide adenine dinucleotide (hereinafter, referred to as NAD⁺). The water used in this case may be in the form of a buffer. Examples of agents used in the buffer include alkali metal salts of phosphoric acid such as sodium phosphate and potassium phosphate, and alkali metal salts of acetic acid sodium acetate and potassium acetate. Examples of alkaline buffer include Tris-HCl buffer, Tris-citrate buffer, and Tris-glycine buffer.

The ALDH step in the first enzymatic method of the present invention may also be carried out by additionally using a hydrophobic organic solvent, i.e. in the presence of water and the hydrophobic organic solvent. Examples of the hydrophobic organic solvent used in this case include esters such as ethyl formate, ethyl acetate, propyl acetate, butyl acetate, ethyl propionate and butyl propionate, alcohols such as n-butyl alcohol, n-amyl alcohol and n-octyl alcohol, aromatic hydrocarbons such as benzene, toluene and xylene, ethers such as diethylether, diisopropylether and methyl-t-butylether), halogenated hydrocarbons such as chloroform and 1,2-dichloroethane, and mixtures thereof.

The ALDH step in the first enzymatic method of the present invention may also be carried out by additionally using a hydrophilic organic solvent, i.e. in the presence of water and an aqueous medium. Examples of the hydrophilic organic solvent used in this case include alcohols such as methanol and ethanol, ketones such as acetone, ethers such as dimethoxyethane, tetrahydrofuran and dioxane, dimethylsulfoxide, and mixtures thereof.

While the ALDH step in the first enzymatic method of the present invention is usually carried out in range of pH of aqueous layer of 3 to 11, the pH may be appropriately changed in such a range that the reaction proceeds. It is preferable that the process of the present invention be carried out in the alkaline range, and it is more preferable that the process be carried out in a range of pH of aqueous layer of 8 to 10.

While the ALDH step in the first enzymatic method of the present invention is usually carried out in a range of about 0° C. to about 60° C., the temperature may be appropriately changed in such a range that the reaction proceeds.

The ALDH step in the first enzymatic method of the present invention is usually carried out in a range of about 0.5 hours to about 10 days. After the completion of adding the sulfur-containing amino alcohol compound represented by the formula (1) [i.e. Compound (1)], which is the starting compound, the endpoint of the reaction can be checked, for example, by measuring the amount of the above corresponding sulfur-containing amino alcohol compound in the reaction solution by liquid chromatography or gas chromatography and others.

The concentration of the above corresponding sulfur-containing α-amino aldehyde compound, which is the starting compound used in the first enzymatic method of the present invention, is usually 50% (w/v) or less and the sulfur-containing α-amino aldehyde compound may be continuously or successively added to a reaction system in order to maintain the concentration of the above corresponding sulfur-containing α-amino aldehyde compound in the reaction system nearly constant.

During the ALDH step in the first enzymatic method of the present invention, a sugar such as glucose, sucrose or fructose, or a surfactant such as Triton X-100 (registered trade mark) or Tween 60 (registered trade mark) may be added to the reaction system as needed.

The ADH/ALDH step in the second enzymatic method of the present invention is usually carried out in the presence of water, preferably, in an additive system with coenzyme such as oxidized nicotinamide adenine dinucleotide (hereinafter, referred to as NAD⁺). The water used in this case may be in the form of a buffer. Examples of buffering agents used in the buffer include alkali metal salts of phosphoric acid such as sodium phosphate and potassium phosphate, and alkali metal salts of acetic acid such as sodium acetate and potassium acetate. Examples of alkaline buffer include Tris-HCl buffer, Tris-citrate buffer, and Tris-glycine buffer.

The ADH/ALDH step in the second enzymatic method of the present invention may also be carried out by additionally using a hydrophobic organic solvent, i.e. in the presence of water and the hydrophobic organic solvent. Examples of the hydrophobic organic solvent used in this case include esters such as ethyl formate, ethyl acetate, propyl acetate, butyl acetate, ethyl propionate and butyl propionate, alcohols such as n-butyl alcohol, n-amyl alcohol and n-octyl alcohol, aromatic hydrocarbons such as benzene, toluene and xylene, ethers such as diethylether, diisopropylether and methyl-t-butylether, halogenated hydrocarbons such as chloroform and 1,2-dichloroethane, and mixtures thereof.

The ADH/ALDH step in the second enzymatic method of the present invention may also be carried out by additionally using a hydrophilic organic solvent, i.e. in the presence of water and an aqueous medium. Examples of the hydrophilic organic solvent used in this case include alcohols such as methanol and ethanol, ketones such as acetone, ethers such as dimethoxyethane, tetrahydrofuran and dioxane, dimethylsulfoxide, and mixtures thereof.

While the ADH/ALDH step in the second enzymatic method of the present invention is usually carried out in range of pH of aqueous layer of 3 to 11, the pH may be appropriately changed in such a range that the reaction proceeds. It is preferable that the process of the present invention be carried out in the alkaline range, and it is more preferable that the process be carried out in a range of pH of aqueous layer of 8 to 10.

While the ADH/ALDH step in the second enzymatic method of the present invention is usually carried out in a range of about 0° C. to about 60° C., the temperature may be appropriately changed in such a range that the reaction proceeds.

The ADH/ALDH step in the second enzymatic method of the present invention is usually carried out in a rage of for about 0.5 hours to about 10 days. After the completion of adding the sulfur-containing amino alcohol compound represented by the formula (1) [i.e. Compound (1)], which is the starting compound, the endpoint of the reaction can be checked, for example, by measuring the amount of the sulfur-containing amino alcohol compound of the formula (1) in the reaction solution or the amount of the above corresponding sulfur-containing α-amino acid compound represented contained in the reaction solution by liquid chromatography or gas chromatography and the others.

The concentration of the sulfur-containing amino alcohol compound represented by the formula (1) [i.e. Compound (1)], which is the starting compound used in the second enzymatic method of the present invention is usually 50% (w/v) or less and the sulfur-containing amino alcohol compound of the formula (1) [i.e. Compound (1)] may be continuously or successively added to a reaction system in order to maintain the concentration of the sulfur-containing amino alcohol compound of the formula (1) in the reaction system nearly constant.

During the ADH/ALDH step in the second enzymatic method of the present invention, for example, a sugar such as glucose, sucrose and fructose or a surfactant such as Triton X-100 (registered trade mark) and Tween 60 (registered trade mark) may be added to the reaction system as needed.

In the enzymatic method of the present invention, the collection of the sulfur-containing α-amino acid compound represented by the formula (2) (i.e. the Compound (2)) from the reaction solution may be carried out by any methods commonly known in the art.

Example of the method include purification by performing post-treatment of the reaction solution such as organic solvent extraction, concentration, ion exchange method and crystallization, if necessary in combination with column chromatography, and distillation and others.

The sulfur-containing amino acid compound represented by the formula (2) prepared by the process of the present invention may be in the form of a salt.

### EXAMPLES

Hereinafter, the present invention is explained in more detail with some examples, which should not be construed to be limited thereto.

### Example 1

### Production of the Plasmid Containing the Present Polynucleotide

DNA fragment comprising the nucleotide sequence of SEQ ID NO: 5 to which NcoI site was added at its 5′-end and XbaI site was added at its 3′-end using Assembly PCR method, was requested artificial gene manufacture (Hokkaido System Science Co., Ltd) to synthesize it. The synthesized DNA fragment were double-digested with two kinds of restriction enzymes (NcoI and XbaI), then the resulting double-digested DNA fragment of about 1.1 kbp was purified.

Separately, plasmid vector pTrc99A (Pharmacia) was double-digested with two kinds of restriction enzymes (NcoI and Xba), then the resulting double-digested DNA fragment was purified.

The resulting two kinds of purified DNA fragments obtained thus were mixed, and then, were ligated with T4 DNA ligase, and thereafter, E. coli DH5α strain were transformed with the resulting ligation liquid.

The resulting transformants were cultured on a LB agar medium containing 50 μg/ml ampicillin, and from grown colonies, 3 colonies were selected randomly. These selected colonies were inoculated to sterilized LB media (2 ml) each containing 50 μg/ml ampicillin, and then cultured in a test tube while shaking (37° C., 24 hours). From each of the cultured microbial cell, plasmids were taken out using QIAprep Spin Miniprep Kit (Qiagen).

Respective parts of the taken out plasmids were double-digested with two kinds of restriction enzymes of NcoI and XbaI, then, the double-digested DNA fragments were subjected to electrophoresis, and it was confirmed that the above-mentioned DNA fragment of about 1.1 kbp was inserted into the taken out every plasmids (hereinafter, this plasmid may be sometimes referred to as plasmid pTrcADH).

Using the resulting plasmid pTrcADH, E. coli DH5α strain was transformed. The resulting transformant was inoculated to sterilized LB medium (100 ml) containing 50 μg/ml ampicillin, and then cultured while shaking (37° C., 24 hours). The resulting culture liquid was centrifugally separated to obtain a microbial cell of the present transformant.

### Example 2

### Production of the Sulfur-Containing α-Amino Acid Compound from the Sulfur-Containing Amino Alcohol Compound Using the E. coli Transformant Producing the Present Enzyme

Each of the present transformant obtained in Example 1 (i.e. E. coli DH5α strain containing plasmid pTrcADH) and E. coli DH5α strain containing plasmid vector pTrc99A (Pharmacia) was inoculated to sterilized LB medium (100 ml) containing 50 μg/ml ampicillin and 0.1 mM IPTG, and then each of these medium was cultured while shaking (30° C., 24 hours). Each of the resulting culture liquid was centrifugally separated to obtain about 0.6 g of each of wet microbial cell. Each of the resulting wet microbial cell was suspended into 10 mL of 0.2 M Tris-HCl buffer (pH 9), and thereafter, the microbial cells in the suspension were disrupted with glass beads to obtain about 6 mL of each of cell-free extract. The respective 1 mL of each of the resulting cell-free extract was mixed to obtain 2 mL of the mixture, and thereto was added 2 mg of DL-methioninol (Sigma), 1 mg of NAD⁺ (Oriental Yeast Co., Ltd), 4 mg of sodium pyruvate (Nacalai Tesque) and 25 unit of lactate dehydrogenase (Oriental Yeast Co., Ltd) and then, the resulting mixture was stirred at 30° C. for 24 hours.

After the completion of the reaction, 0.6 ml of the reaction solution was sampled. The suspended solids were removed from the sampling solution, and then, the amount of the produced methionine was analyzed by liquid chromatography. As the result, it was confirmed that methionine was produced in 14.3% yield based on an amount of DL-methioninol used in the reaction. The result of control experiment was described in Example 3.

### Conditions for Content Analysis

Column: Cadenza CD-C18 (4.6 mmφ×15 cm, 3 μm) (manufactured Imtakt Corp.)
Mobile phase: 0.1% aqueous trifluoroacetic acid as
Solution A-, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Example 3

### Production of the Sulfur-Containing α-Amino Acid Compound from the Sulfur-Containing Amino Alcohol Compound Using the E. coli Transformant Producing the Present Enzyme

### Control Experiment

E. coli DH5α strain containing plasmid vector pTrc99A (Pharmacia) was inoculated to sterilized LB medium (100 ml) containing 50 μg/ml ampicillin and 0.1 mM IPTG, and then, this medium were cultured while shaking (30° C., 24 hours). The resulting culture liquid was centrifugally separated to obtain about 0.6 g of wet microbial cell. The resulting wet microbial cell was suspended into 10 mL of 0.2 M Tris-HCl buffer (pH 9), and thereafter, the microbial cell in the suspension was disrupted with glass beads to obtain about 6 mL of cell-free extract. To 2 mL of the resulting cell-free extract was added 2 mg of DL-methioninol (Sigma), 1 mg of NAD⁺ (Oriental Yeast Co., Ltd), 4 mg of sodium pyruvate (Nacalai Tesque) and 0.25 unit of lactate dehydrogenase (Oriental Yeast Co., Ltd) and then, the resulting mixture was stirred at 30° C. for 24 hours.

After the completion of the reaction, 0.6 ml of the reaction solution was sampled. The suspended solids were removed from the sampling solution, and then the amount of the produced methionine was analyzed by liquid chromatography. As the result, it was confirmed that DL-methioninol used in the reaction was remained in 100%.

### Conditions for Content Analysis

Column: Cadenza CD-C18 (4.6 mmφ×15 cm, 3 μm) (manufactured by Imtakt Corp.)

Mobile phase: 0.1% aqueous trifluoroacetic acid as
Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Example 4

### Production of Plasmid Containing the Present Polynucleotide

Forward primers and Reverse primers that are correspond to an amino acid sequence of SEQ ID NO: 1, are cut, divided in about 40 bp length to synthesize thirty (30) primers thereof respectively. Assembly PCR method is performed with the synthesized primers under the following condition.

### [Reaction Solution Composition]

dNTP (each 2.5 mM-mix) 1 μL
primer mix (250 μM) 0.5 μL
5× buffer (with MgCl) 10 μL
enz.expandHiFi (5 U/μL) 0.5 μl
ultrapure water 38 μl

A vessel containing a reaction solution of the above-mentioned composition is set on PERKIN ELMER-GeneAmp PCR System 9700, and then, a cycle of 94° C. (for 30 seconds)-52° C. (for 30 seconds)-68° C. (for 30 seconds) is repeated 55 times.

After completion of the reaction, PCR reaction is carried out again with the reaction solution as a template under the following reaction condition. After heating at 94° C. (for 2 minutes), a cycle of 94° C. (for 30 seconds)-53° C. (for 30 seconds)-68° C. (for 1.5 minutes) is repeated 30 times. In the reaction, oligonucleotide primer comprising the nucleotide sequence set forth in SEQ ID NO: 6 to which NcoI site is added at 5′-end, and oligonucleotide primer comprising the nucleotide sequence set forth in SEQ ID NO: 7 to which XbaI site is added at 5′-end, are used.

### [Reaction Solution Composition]

template 1 μL
dNTP (each 2.5 mM-mix) 1 μL
primer mix (250 μM) 0.5 μL
5× buffer (with MgCl) 10 μL
enz.expandHiFi (5 U/μL) 0.5 μl
ultrapure water 36.5 μl

Thereafter, a part of the PCR reaction solution is collected and purified on agarose gel to obtain a DNA fragment, and thereto is added two kinds of restriction enzymes (NcoI and XbaI) to double-digested a DNA fragment of about 1.1 kbp. Then the resulting double-digested DNA fragment of about 1.1 kbp is purified.

Separately, plasmid vector pTrc99A (Pharmacia) is double-digested with two kinds of restriction enzymes (NcoI and Xba), then the resulting double-digested DNA fragment is purified.

The resulting two kinds of purified DNA fragments thus obtained are mixed, and are ligated with T4 DNA ligase, and then E. Coli DH5α strain are transformed with the resulting ligation liquid.

The resulting transformants are cultured on a LB agar medium containing 50 μg/ml ampicillin, and from grown colonies, 3 colonies are selected randomly. These selected colonies are inoculated to sterilized LB medium (2 ml) each containing 50 μg/ml ampicillin, and then, the medium are cultured in a test tube while shaking (37° C., 24 hours). From the each cultured microbial cell, plasmids are taken out using QIAprep Spin Miniprep Kit (Qiagen).

Respective parts of the taken out plasmids are double-digested with two kinds of restriction enzymes of NcoI and XbaI, then, the double-digested DNA fragments are subjected to electrophoresis, and it is confirmed that the above-mentioned DNA fragment of about 1.1 kbp is inserted into every the taken out plasmids (hereinafter, this plasmid may be sometimes referred to as plasmid pTrcADH).

Using the resulting plasmid pTrcADH, E. coli DH5α strain is transformed. The resulting transformant is inoculated to sterilized LB medium (100 ml) containing 50 μg/ml ampicillin, and then the medium are cultured while shaking (37° C., 24 hours). The resulting culture liquid is centrifugally separated to obtain a microbial cell of the present transformant.

### Example 5

### Production of the Sulfur-Containing α-Amino Acid Compound from the Sulfur-Containing Amino Alcohol Compound According to the Enzymatic Method of the Present Invention

In a screw-top test tube is placed 2 ml of 0.1 M Tris-hydrochloride buffer (pH 9), and thereto was added a) alcohol dehydrogenase (from horse liver) (Sigma) and b) aldehyde dehydrogenase (from yeast) (Sigma) as the present enzyme, and further added 4 mg of sodium pyruvate (Nacalai Tesque), 0.6 mg of NAD⁺ and 0.25 mg of lactate dehydrogenase (Wako Pure Chemicals Industries, Ltd.) and then the reaction solution was mixed. To the mixture was added 2 mg of DL-methioninol (Sigma) as a starting compound, and then the resulting mixture was shaken at 30° C. for 22 hours.

After the completion of the reaction, 0.5 ml of the reaction solution was sampled. The amount of the produced methionine in the sample solution was analyzed by liquid chromatography. As the result, methionine was obtained in yield 23%.

### Conditions for Content Analysis

Column: Cadenza CD-C18 (4.6 mmφ×15 cm, 3 μm) (manufactured by Imtakt Corp.)
Mobile phase: 0.1% trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Example 6

### Production of the Sulfur-Containing α-Amino Acid Compound from the Sulfur-Containing Amino Alcohol Compound According to the Enzymatic Method of the Present Invention

In a screw-top test tube is placed 2 ml of 0.1 M Tris-hydrochloride buffer (pH 9), and thereto was added alcohol dehydrogenase (from horse liver) (Sigma) as the present enzyme, and further added 4 mg of sodium pyruvate (Nacalai Tesque), 0.6 mg of NAD⁺ and 0.25 mg of lactate dehydrogenase (Wako Pure Chemicals Industries, Ltd.) and then the reaction solution was mixed. To the mixture was added 2 mg of DL-methioninol (Sigma) as a starting compound, and then the resulting mixture was shaken at 30° C. for 22 hours.

After the completion of the reaction, 0.5 ml of the reaction solution was sampled. The amount of the produced methionine in the sample solution was analyzed by liquid chromatography. As the result, methionine was obtained in yield 17%.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Reference Example 1

### Screening Microorganisms Capable of Converting the Sulfur-Containing Amino Alcohol Compound into a Corresponding Sulfur-Containing α-Amino Aldehyde Compound

In a test tube is placed 5 ml of sterilized culture medium, which is prepared by adding polypeptone (5 g), yeast extracts (3 g), meat extracts (3 g), ammonium sulfate (0.2 g), potassium dihydrogen phosphate (1 g) and magnesium sulfate heptahydrate (0.5 g) to 1 L of water, and then adjusting the pH to 7.0, and thereto is inoculated with microorganism obtained by purchasing from the previously mentioned culture collection or a microorganism isolated from soils. The resulting is incubated with shaking at 30° C. under an aerobic condition. After the completion of the incubation, the microbial cells are collected by centrifugation to obtain viable cells. In a screw-top test tube is placed 2 ml of 0.1 M Tris-glycine buffer (pH 10), and thereto is added the above-prepared viable cells, and then the mixture is suspended. To the suspension is added 2 mg of methioninol, and then the resulting mixture is shaken at 30° C. for 3 to 7 days.

After the completion of the reaction, 1 ml of the reaction solution is sampled. The cells are removed from the sampling solution, and then the amount of the produced 1-amino-3-(methylthio)propionaldehyde is analyzed by liquid chromatography.

Thus, microorganisms capable of converting the sulfur-containing amino alcohol compound into the corresponding sulfur-containing α-amino aldehyde compound are screened.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Reference Example 2

### Screening Microorganisms Capable of Converting the Sulfur-Containing α-Amino Aldehyde Compound into a Corresponding Sulfur-Containing α-Amino Acid Compound

In a test tube is placed 5 ml of sterilized culture medium, which is prepared by adding polypeptone (5 g), yeast extracts (3 g), meat extracts (3 g), ammonium sulfate (0.2 g), potassium dihydrogen phosphate (1 g) and magnesium sulfate heptahydrate (0.5 g) to 1 L of water, and then adjusting the pH to 7.0, and thereto is inoculated with a microorganism obtained by purchasing from the previously mentioned culture collection or a microorganism isolated from soils. The resulting is incubated with shaking at 30° C. under an aerobic condition. After the completion of the incubation, the microbial cells are collected by centrifugation to obtain a viable microbial cell. In a screw-top test tube is placed 2 ml of 0.1 M Tris-glycine buffer (pH 10), and thereto is added the above-prepared viable microbial cell, and then the mixture solution is suspended. To the suspension is added 2 mg of 1-amino-3-(methylthio)propionaldehyde, and then the resulting mixture is shaken at 30° C. for 3 to 7 days.

After the completion of the reaction, 1 ml of the reaction solution is sampled. The cells are removed from the sampling solution, and then the amount of the produced methionine is analyzed by liquid chromatography.

Thus, microorganisms capable of converting the sulfur-containing α-amino aldehyde compound into the corresponding sulfur-containing α-amino acid compound are screened.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and -methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### Reference Example 3

### Screening Microorganisms Capable of Converting the Sulfur-Containing Amino Alcohol Compound into a Corresponding Sulfur-Containing α-Amino Acid Compound

In a test tube is placed 5 ml of sterilized culture medium, which is prepared by adding polypeptone (5 g), yeast extracts (3 g), meat extracts (3 g), ammonium sulfate (0.2 g), potassium dihydrogen phosphate (1 g) and magnesium sulfate heptahydrate (0.5 g) to 1 L of water, and then adjusting the pH to 7.0, and thereto is inoculated with a microorganism obtained by purchasing from the previously mentioned culture collection or a microorganism isolated from soils. The resulting is incubated with shaking at 30° C. under an aerobic condition. After the completion of the incubation, the microbial cells are collected by centrifugation to obtain viable cells. In a screw-top test tube is placed 2 ml of 0.1 M Tris-glycine buffer (pH 10), and thereto is added the above-prepared viable cells, and then the mixture is suspended. To the suspension is added mg of methioninol, and then the resulting mixture is shaken at 30° C. for 3 to 7 days.

After the completion of the reaction, 1 ml of the reaction solution is sampled. The cells are removed from the sampling solution, and then the amount of the produced methionine is analyzed by liquid chromatography.

Thus, microorganisms capable of converting the sulfur-containing amino alcohol compound into the corresponding sulfur-containing α-amino acid compound are screened.

### Conditions for Content Analysis

Column: Cadenza CD-C18 (4.6 mmφ×15 cm, 3 μm) (manufactured by Imtakt Corp.)
Mobile phase: 0.1% aqueous trifluoroacetic acid as Solution A, and methanol as Solution B

| Time (minutes) | Solution A (%):Solution B (%) |
|---|---|
| 0 | 100:0 |
| 10 | 100:0 |
| 20 | 50:50 |
| 25 | 50:50 |
| 25.1 | 100:0 |

Flow rate: 0.5 ml/min
Column temperature: 40° C.

### Detection: 220 nm

### INDUSTRIAL APPLICABILITY

The present invention can provide a novel process for producing a sulfur-containing α-amino acid compound such as methionine.

1. A process for producing a sulfur-containing α-amino acid compound represented by the formula (2): 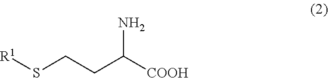
wherein R¹ represents hydrogen, an alkyl group having 1 to 8 carbon atoms, or an aryl group having 6 to 20 carbon atoms;
comprising
a step of reacting a compound represented by the formula (1): 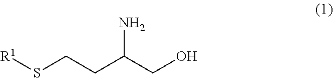
wherein R¹ is the same as defined above;
with a microbial cell or a processed product of the microbial cell of any of the following transformants:
(a) both a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound and a transformant in which has been introduced into a microorganism cell a polynucleotide comprising a nucleotide sequence encoding an amino acid sequence of an enzyme that is capable of converting the above corresponding sulfur-containing α-amino aldehyde compound into a corresponding sulfur-containing α-amino acid compound,
(b) a transformant in which have been introduced into a microorganism both a polynucleotide comprising a nucleotide sequence encoding an amino acid sequence of an enzyme that is capable of converting the above sulfur-containing amino alcohol compound into a corresponding sulfur-containing α-amino aldehyde compound and a polynucleotide comprising a nucleotide sequence encoding an amino acid sequence of an enzyme that is capable of converting the above corresponding sulfur-containing α-amino aldehyde compound into a corresponding sulfur-containing α-amino acid compound or
(c) a transformant in which have been introduced into a microorganism a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

2. The process according to claim 1 wherein the transformant is the transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, wherein said enzyme comprises any of the following amino acid sequences:
a) the amino acid sequence of SEQ ID NO: 1,
b) an amino acid sequence which is encoded by a nucleotide sequence of a DNA that hybridizes to a DNA consisting of the nucleotide sequence of SEQ ID NO: 2 or 5 under a stringent condition and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, or
c) an amino acid sequence in which one or a plurality of amino acids have been deleted, substituted or added in the amino acid sequence of SEQ ID NO: 1 and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

3. The process according to claim 1 or 2 wherein R¹ of the sulfur-containing amino alcohol compound and the sulfur-containing α-amino acid compound is an alkyl group having 1 to 5 carbon atoms.

4. The process according to claim 1 or 2 wherein R¹ of the sulfur-containing amino alcohol compound and the sulfur-containing α-amino acid compound is a methyl group.

5. A recombinant vector comprising a DNA in which a promoter functional in a microorganism cell is operably linked to a polynucleotide comprising a nucleotide sequence encoding an amino acid sequence of an enzyme that is capable of converting the above sulfur-containing amino alcohol compound represented by the formula (1): 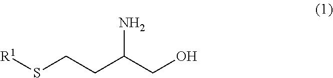
wherein R¹ represents hydrogen, an alkyl group having 1 to 8 carbon atoms, or an aryl group having 6 to 20 carbon atoms;
into a corresponding sulfur-containing α-amino aldehyde compound as well as capable of converting the above sulfur-containing α-amino aldehyde compound into a corresponding sulfur-containing α-amino acid compound.

6. The vector according to claim 5 wherein the polynucleotide comprises the nucleotide sequence of SEQ ID NO: 5.

7. A transformant in which the recombinant vector according to claim 5 or 6 has been introduced into a microorganism cell.

8. The transformant according to claim 7 wherein the microorganism cell is an E. coli cell.

9. A process for producing a transformant comprising a step of introducing the vector according to claim 5 or 6 into a microorganism cell.

10. A use of a microbial cell or a processed product of the microbial cell of any of the following transformants as a catalyst for converting a sulfur-containing amino alcohol compound represented by the formula (1): 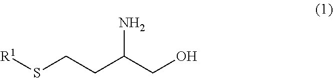
wherein R¹ represents hydrogen, an alkyl group having 1 to 8 carbon atoms, or an aryl group having 6 to 20 carbon atoms;
into a corresponding sulfur-containing α-amino acid compound:
(a) both a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound and a transformant in which has been introduced into a microorganism cell a polynucleotide comprising a nucleotide sequence encoding an amino acid sequence of an enzyme that is capable of converting the above corresponding sulfur-containing α-amino aldehyde compound into a corresponding sulfur-containing α-amino acid compound, into a microbial cell,
(b) a transformant in which have been introduced into a microorganism cell both a polynucleotide comprising a nucleotide sequence encoding an amino acid sequence of an enzyme that is capable of converting the above sulfur-containing amino alcohol compound into a corresponding sulfur-containing α-amino aldehyde compound and a polynucleotide comprising a nucleotide sequence encoding an amino acid sequence of an enzyme that is capable of converting the above corresponding sulfur-containing α-amino aldehyde compound into a corresponding sulfur-containing α-amino acid compound, or
(c) a transformant in which have been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

11. The use according to claim 10 wherein the transformant is a transformant in which has been introduced into a microorganism cell a polynucleotide comprising a nucleotide sequence encoding an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, wherein said enzyme comprises any of the following amino acid sequences:
a) the amino acid sequence set forth in SEQ ID NO: 1,
b) an amino acid sequence which is encoded by a nucleotide sequence of a DNA that hybridizes to a DNA consisting of the nucleotide sequence of SEQ ID NO: 2 or 5 under a stringent condition and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound, or
c) an amino acid sequence in which one or a plurality of amino acids have been deleted, substituted or added in the amino acid sequence of SEQ ID NO: 1 and which is an amino acid sequence of an enzyme that is capable of converting the above sulfur-containing amino alcohol compound into a corresponding sulfur-containing α-amino aldehyde compound as well as capable of converting the above sulfur-containing α-amino aldehyde compound into a corresponding sulfur-containing α-amino acid compound.

12. The use according to claim 10 or 11 wherein the transformant is a transformant in which has been introduced into a microorganism cell a polynucleotide comprising a nucleotide sequence encoding the amino acid sequence of SEQ ID NO: 1.

13. The use according to claim 10 or 11 wherein R¹ of the sulfur-containing amino alcohol compound is an alkyl group having 1 to 5 carbon atoms.

14. The use according to claim 10 or 11 wherein R¹ of the sulfur-containing amino alcohol compound is a methyl group.

15. A process for producing a sulfur-containing α-amino acid compound represented by the formula (2): 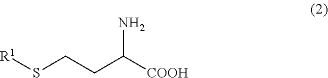
wherein R¹ represents hydrogen, an alkyl group having 1 to 8 carbon atoms, or an aryl group having 6 to 20 carbon atoms;
comprising
a step of reacting a sulfur-containing amino alcohol compound represented by the formula (1): 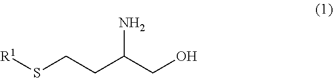
wherein R¹ is the same as defined above;
with an enzyme that is capable of converting the above sulfur-containing amino alcohol compound into a corresponding sulfur-containing α-amino aldehyde compound as well as capable of converting t the above sulfur-containing α-amino aldehyde compound into a corresponding sulfur-containing α-amino acid compound.

16. A process for producing a sulfur-containing α-amino acid compound represented by the formula (2): 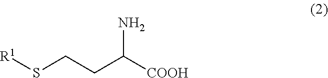
wherein R¹ represents hydrogen, an alkyl group having 1 to 8 carbon atoms, or an aryl group having 6 to 20 carbon atoms;
comprising
a step of reacting a sulfur-containing amino alcohol compound represented by the formula (1): 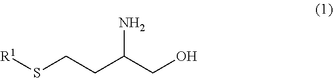
wherein R¹ is the same as defined above;
with an enzyme capable of converting the above sulfur-containing amino alcohol compound into a corresponding sulfur-containing α-amino aldehyde compound, and
a step of reacting the sulfur-containing α-amino aldehyde compound obtained in the above step with an enzyme capable of converting the above sulfur-containing α-amino aldehyde compound into a corresponding sulfur-containing α-amino acid compound.

17. The process according to claim 15 or 16 wherein R¹ of the sulfur-containing amino alcohol compound and the sulfur-containing α-amino acid compound is an alkyl group having 1 to 5 carbon atoms.

18. The process according to claim 15 or 16 wherein R¹ of the sulfur-containing amino alcohol compound and the sulfur-containing α-amino acid compound is a methyl group.


SEQUENCE LISTING (7 sequences)

SEQ ID NO: 1 - protein, 375 aa, Equus caballus
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 2 - DNA, 1128 bp, Equus caballus
atgagcacagcaggaaaagtaataaaatgcaaagcggctgtgctgtgggaggaaaagaaaccattttccatcgaggaggtggaggttgcacccccgaaggcccatgaagtccgtataaagatggtggccacaggaatttgtcgctcagatgaccacgtggttagtggaacccttgtcacacctcttcctgtgatcgcaggccatgaggcagcgggcattgtggagagcattggagaaggcgtcactacagtaagaccaggtgataaagtcatcccactctttactccccagtgtggaaaatgcagggtttgtaagcaccctgaaggcaacttctgcttgaaaaatgatctgagcatgcctcggggaaccatgcaggatggtaccagcaggttcacctgcagagggaagcccatccaccacttccttggcaccagcaccttctcccagtacaccgtggtggacgagatctcagtggccaagatcgatgcggcctcaccgctggagaaagtctgtctcattggctgtggattttctactggttatgggtctgcagtcaaggttgccaaggtcacccagggctccacctgtgccgtgtttggccttggaggagtgggcctgtctgttatcatgggctgtaaagcagccggagcggccaggatcattggggtggacatcaacaaagacaagtttgcaaaggccaaagaagtgggtgccactgagtgtgtcaaccctcaggactacaagaaacccatccaggaggtgctgacagaaatgagcaatggaggtgtggatttttcctttgaagtcattggtcggctcgacactatggtgactgccttgtcatgctgtcaagaagcatatggtgtgagcgtcattgtgggagtacctcctgattcccaaaatctctctatgaatcctatgttgctactgagtggacgtacctggaaaggagctatttttggcggttttaagagtaaagattctgtccccaaacttgtggccgattttatggctaaaaagtttgcactggatcctttaatcacccatgttttaccttttgaaaaaataaatgaaggatttgacctgcttcgctctggagagagtatccgtaccatcctgacgttttga

SEQ ID NO: 3 - DNA, 30 bp, Artificial Sequence
  Designed oligonucleotide primer for PCR
atgagcacagcaggaaaagtaataaaatgc

SEQ ID NO: 4 - DNA, 30 bp, Artificial Sequence
  Designed oligonucleotide primer for PCR
tcaaaacgtcaggatggtacggatactctc

SEQ ID NO: 5 - DNA, 1128 bp, Artificial Sequence
  Artificial synthesized polynucleotide having a nucleotide sequence designed by using codons suitable for E. coli
atgtctactgcgggtaaagtaattaaatgcaaggctgctgtgctgtgggaagagaagaaaccgttctccatcgaagaagttgaggtagccccgccgaaagcgcacgaagttcgcatcaaaatggtggcgaccggtatttgtcgtagcgacgatcacgttgtgtccggtaccctggtaaccccgctgccggttatcgctggccatgaagcagctggcatcgttgaatctatcggcgaaggcgtaactaccgtacgcccaggtgacaaagttatcccgctgtttacgccgcaatgcggtaaatgccgtgtctgtaaacacccggaaggcaacttctgcctgaaaaatgatctgtctatgcctcgcggcacgatgcaagacggtaccagccgtttcacctgccgtggcaaaccaatccaccacttcctgggtactagcaccttctctcagtacaccgttgttgacgaaatctccgttgcgaagattgacgcggcgtccccgctggagaaagtgtgcctgatcggctgtggtttctccacgggttatggttccgcagttaaagttgcgaaagttacccagggctctacttgcgcggtgttcggcctgggtggtgtgggtctgtccgttatcatgggttgcaaagcagccggtgctgctcgtatcattggtgtggatattaacaaagataagtttgcaaaagctaaagaggtcggtgccaccgaatgtgtaaacccgcaggactacaaaaagccaatccaggaagtactgaccgaaatgtccaacggtggtgtcgacttctcttttgaagtaatcggccgtctggatactatggtgaccgcactgtcctgctgtcaggaagcgtacggcgtgagcgtcatcgttggcgtgccgccggacagccagaacctgtctatgaaccctatgctgctgctgtctggccgtacttggaaaggcgcaatttttggcggcttcaaatctaaagatagcgttccgaaactggtggccgattttatggctaagaaattcgcactggacccgctgatcactcatgttctgccgttcgagaaaatcaacgaaggcttcgatctgctgcgttctggcgaatccattcgcaccattctgactttctaa

SEQ ID NO: 6 - DNA, 30 bp, Artificial Sequence
  Designed oligonucleotide primer for PCR
atgtctactgcgggtaaagtaattaaatgc

SEQ ID NO: 7 - DNA, 33 bp, Artificial Sequence
  Designed oligonucleotide primer for PCR
ttagaaagtcagaatggtgcgaatggattcgcc